(19) United States
(12) Patent Application Publication
(10) Pub. No.: US 2024/0423661 A1
(43) Pub. Date: Dec. 26, 2024

(54) DEVICES AND SYSTEMS FOR REMOVING TISSUE ABOVE AND /OR DRAINING A SUBCUTANEOUS SKIN ABSCESS

(71) Applicant: INOVA MEDICAL PTY LTD, Perth (AU)

(72) Inventors: Melanie Kathryn WHITE, Perth (AU); Alex John Cullen HAYES, Perth (AU); Ming Khoon YEW, Perth (AU); Giles MATTHEWS, Cremorne (AU); Michael WESTWOOD, Cremorne (AU); Louis MILLS, Cremorne (AU)

(73) Assignee: INOVA MEDICAL PTY LTD, Perth (AU)

(21) Appl. No.: 18/707,518

(22) PCT Filed: Nov. 4, 2022
(86) PCT No.: PCT/AU2022/051329

§ 371 Date: May 3, 2024

(30) Foreign Application Priority Data
Nov. 5, 2021 (AU) 2021903549

(51) Int. Cl. A61B 17/3205; A61B 17/00; A61B 17/32; A61B 17/34

(52) U.S. Cl. CPC A61B 17/32053; A61B 17/00234; A61B 17/3421; A61B 2017/00353; A61B 2017/00367; A61B 2017/00477; A61B 2017/00561; A61B 2017/320064; A61B 2217/005

(57) ABSTRACT

A device for removing tissue above a subcutaneous skin abscess and/or draining a subcutaneous skin abscess. The device comprises a tissue engagement component, a container configured to contain at least part of the tissue engagement component, and a cutting element configured to cut tissue of a patient. The tissue engagement component is configured to engage the tissue cut by the cutting element such that movement of the tissue engagement component into a containment volume of the container causes movement of at least part of the tissue cut by the cutting element into the containment volume.

### CROSS-REFERENCE TO RELATED APPLICATIONS

This application claims priority to Australian provisional patent application No. 2021903549 filed on 5 Nov. 2021, the disclosure of which is incorporated herein by reference in its entirety.

### TECHNICAL FIELD

Some embodiments relate to a device or system for removing tissue above and/or draining a subcutaneous skin abscess. Some embodiments of the present disclosure generally relate to a device for retrieving tissue. In particular, some embodiments of the present disclosure relate to a device for retrieving a portion of the skin of a patient. Some embodiments of the present disclosure generally relate to a percutaneous drainage device. In particular, some embodiments of the present disclosure relate to a device for accessing and draining a fluid collection located under or within the skin of a patient.

### BACKGROUND

Each year a large number (e.g. several million) of patients present to emergency rooms seeking medical assistance for skin and soft tissue infections. A significant number of these infections are subcutaneous abscesses (also referred to as subcutaneous skin abscesses), which are confined accumulations or collections of pus and dead tissue below the skin. Patients presenting with this common skin condition usually experience pain and can also present with fever or chills.

The treatment of subcutaneous collections of fluids, such as skin abscesses, is a commonly performed medical procedure. Some abscesses are ‘simple’ or ‘pointing’, and are almost ready to burst through the skin. These abscesses can typically be treated in an outpatient setting with local anaesthetic via an incision and drainage procedure. After administration of a local anaesthetic, a small incision is made at the site to puncture the skin. Contents of the abscess can then escape by draining out through the incision, sometimes assisted by manual expression of the contents by a clinician. Contents of the abscess may project upward and outward when excised and clinicians typically use personal protective equipment to mitigate risk of self contamination.

Presently, many complex and/or deep (more than 2 cm below surface of the skin) skin abscess cases presenting to hospitals are treated in theatre with surgery and under a general anaesthetic. Whilst surgical treatment generally resolves the problem of the abscess, it generates a series of new problems. Patients undergoing surgical abscess treatment typically require a multi-day hospital stay. They must be fasted prior to surgery, and face the risks associated with administration of general anaesthesia. Surgical treatment of abscesses requires an incision that needs to be long and deep enough to allow access to the abscess cavity. Typically, the incision will extend across the entire diameter of the abscess and, consequently, scarring may become of concern to the patient. Further, the use of longitudinal force to penetrate skin and tissue to reach the abscess carries a risk of inadvertently puncturing underlying tissue structures.

Surgical treatment of abscesses is thus financially costly for the relevant health care system and patient, and also carries risks associated with surgery and administration of general anaesthesia.

Other medical procedures may similarly require access to a fluid collection site in the body of a patient to drain a fluid, which may be liquid or gas. At least some of these medical procedures share similar problems and risks to deep skin abscess drainage procedures.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

According to a first aspect of the present disclosure, there is provided a device for removing tissue above a subcutaneous skin abscess. The device may comprise: a tissue engagement component, comprising: a penetration portion; a barb; and a shaft; a container configured to contain at least part of the tissue engagement component; and a cutting element configured to cut tissue of a patient; wherein the tissue engagement component is configured to engage the tissue cut by the cutting element such that movement of the tissue engagement component into a containment volume of the container causes movement of at least part of the tissue cut by the cutting element into the containment volume.

In some embodiments, the cutting element is configured to cut through the skin surface of a patient to cut out a skin plug from epidermal and dermal tissue of a patient. In some embodiments the tissue engagement component is configured to engage the skin plug cut by the cutting element such that movement of the tissue engagement component into a containment volume of the container causes movement of at least part of the skin plug cut by the cutting element into the containment volume.

In some embodiments the container has a proximal end and a distal end and the cutting element has a cutting position in which the cutting element projects outward from a distal end of the container. The cutting element may have a cutting edge at the distal end for cutting through the skin surface and into the epidermal and dermal layers of the skin. The cutting element may present a level cutting plane. In some examples, the cutting element may be cylindrical. In some examples the cutting element defines a cutting lumen through which the tissue engagement component may project.

The cutting element may be used to cut around a portion of epidermal and dermal tissue to form a skin plug which may be extracted by the tissue engagement component. In some examples the cutting element has a length of 4 mm to 10 mm, in some examples 5 mm to 10 mm, in some examples 5 mm to 8 mm and in some examples of about 7 mm. Where the cutting element defines a cutting lumen, the diameter of the cutting lumen defines the dimensions of the skin plug. In some examples the cutting element has an outer diameter of 3 mm-6 mm, in other examples of 4 mm-6 mm, in other examples of 3 mm to 5.5 mm, in still other examples of 4.5 mm to 5.5 mm and in some examples of about 5 mm.

In some embodiments, the tissue engagement component shaft may have a diameter of 1 mm to 3 mm. In some embodiments a clearance between the tissue engagement component and the inner diameter of the cutting element may be at least 1 mm, in some examples at least 2 mm, in some examples 1 mm to 3 mm, in other examples 2 mm to 3 mm, and in still other examples 2 mm to 2.5 mm. As there is a clearance between the tissue engagement component and the cutting element, the tissue engagement component may retrieve into the container a skin plug having a diameter or dimensions larger than a diameter of the tissue engagement component.

The tissue engagement component may have a harpoon shape. The tissue engagement element has a shaft, a penetration portion and at least one barb. The penetration portion may be a sharp point or a tapered blade at a distal end of the shaft. The barb may be configured to engage with the tissue penetrated by the penetration portion and to pull the skin plug away from the surrounding tissue when the skin plug is retracted. The (or each) barb may be angled away from the penetration portion at the distal end and may extend radially outwards and towards the proximal end. The (or each) barb may be configured to hook and retain tissue when the tissue engagement portion is retracted and moved towards the proximal end of the container. The (or each) barb may be formed integrally with the shaft of the tissue engagement portion. The (or each) barb may be positioned behind the penetration portion, for instance within 3 mm to 1.5 cm of a pointed distal end of the penetration portion.

The (or each) barb may have one or more edges facing away from penetration portion towards a proximal end of the tissue engagement component. In some examples, the barb may be formed by a spike or hook like projection from the shaft, such as a projection pointing towards the distal end of the shaft. In other examples, the barb may be formed by a recess in the body of the shaft. The barb acts to catch and pull on epidermal an dermal tissue when the tissue engagement portion is retracted into the container. The barb may thus engage with and retrieve a skin plug which has been cut by the cutting element. The device is designed so that the cutting element cuts the skin, epidermal and dermal layers to form a skin plug before, or at the same time as, the tissue engagement portion engages with the skin plug. In this way the connection of the skin plug to tissue in the epidermal an dermal layers may be severed by the cutting element; this allows the skin plug to be pulled out by retraction of the tissue engagement component (e.g. by the barb which engages with the skin plug) as the pull of the tissue engagement component is stronger than the connection to the tissue (e.g. subcutaneous fat) beneath the skin plug. The device thus has a ‘cut then grab’ approach. This is in contrast to other types of device, such as morcellators which grab tissue and pull the tissue into a container and only sever the connection after the tissue has been removed from the body.

In some examples the barb is formed by a recess in the tissue retrieval component shaft. In some examples, the shaft has an internal lumen and the recess cuts across the lumen, thereby creating a barb comprising sharp edges facing backwards towards the proximal end. This provides a particularly effective structure for engaging with and retrieving tissue.

In some embodiments, the tissue engagement component defines a tissue engagement component lumen that extends between a first tissue engagement component opening and a second tissue engagement component opening.

In some embodiments, the tissue engagement component defines a barb opening that is fluidly connected to the tissue engagement component lumen.

In some embodiments, the device further comprises a plunger configured move in a longitudinal direction of the container, wherein: the container is configured to contain at least part of the plunger; and movement of the plunger causes movement of the tissue engagement component.

In some embodiments, movement of the plunger causes movement of the cutting element.

In some embodiments, the tissue engagement component is configured to connect to the plunger; the cutting element is configured to connect to the plunger; and the tissue engagement component and the cutting element are separate components that are independently connectable to the plunger.

In some embodiments, the plunger is configured to be moved between: a first position where at least part of the tissue engagement component and at least part of the cutting element are exposed; and a second position where the tissue engagement component and the cutting element are contained within the containment volume of the container.

In some embodiments, the cutting element comprises a cylindrical cutting sharp; and the cutting element extends from a cutting end portion to a second end portion to define a cutting lumen.

In some embodiments, the tissue engagement component extends through the cutting lumen such that the penetration portion and the barb are exposed beyond the cutting lumen.

In some embodiments, the container defines the containment volume and a distal volume; the container comprises a container rim that extends inwardly in a radial direction of the container to define an inner container hole; the container rim is between the containment volume and the distal volume; and the inner container hole fluidly connects the containment volume and the distal volume.

In some embodiments there may be a single plunger. In other embodiments, the plunger is a first plunger; and the tissue retrieval device further comprises a second plunger configured to connect to the first plunger such that, when connected, movement of the second plunger causes corresponding movement of the first plunger; wherein the second plunger is configured to be moved between: an initial position where the second plunger is connected to a first plunger connection portion of the first plunger; and a plunger connection position where the second plunger is connected to a second plunger connection portion of the first plunger; and the plunger connection position and a retracted position.

In some embodiments, the second plunger comprises a second plunger connector configured to enable connection of the second plunger and the first plunger.

In some embodiments, the second plunger connector comprises a second plunger connector member.

In some embodiments, the first plunger connection portion defines a first plunger connection hole that is configured to receive a portion of the second plunger connector member when the second plunger is in the initial position; and the second plunger connection portion defines a second plunger connection hole that is configured to receive the portion of the second plunger connector member when the second plunger is in the plunger connection position.

In some embodiments, the container comprises: a container body lumen extending between a first container body opening and a second container body opening; and a first cap defining a first container outlet configured to receive the tissue engagement component and the cutting element such that at least part of the tissue engagement component and at least part of the cutting element extend outside of the container through the first container outlet, wherein the first cap is configured to be removably connectable to the container body.

In some embodiments, the cutting element is configured to extend through the first container outlet.

In some embodiments, the tissue retrieval device further comprises: a first container cap configured to removably connect to the container body to obstruct the first container body opening; and a second container cap configured to removably connect to the container body to obstruct the second container body opening.

In some embodiments, the second plunger comprises: an intermediate portion; a distal end portion; and a step where the intermediate portion meets the distal end portion.

In some embodiments, an outer dimension of the intermediate portion is larger than an outer dimension of the distal end portion to define the step.

In some embodiments, the second plunger comprises an angled wall, and a plane tangential to a surface of the angled wall is transverse to a longitudinal direction of the second plunger.

In some embodiments, the tissue retrieval device further comprises a plunger sleeve comprising: a first sleeve connector member; and a second sleeve connector member; wherein the plunger sleeve is configured to contain at least part of the first plunger and at least part of the second plunger within a plunger sleeve volume.

In some embodiments, the container is configured to contain the plunger sleeve within the containment volume. The plunger sleeve may receive the plunger. In some cases the plunger sleeve may receive the cutting element and/or the tissue engagement component when they are retracted into the container. The plunger or a sealing component connected to the plunger may form a seal with the plunger sleeve, so as to generate a negative pressure when the plunger is retracted and thereby promote suction of fluid or abscess contents into the container. The plunger sleeve may include a locking mechanism to releasably lock the plunger, tissue engagement component and/or cutting element in a cutting state in which the tissue engagement component and/or cutting element are fixed relative to the container and project from the distal end of the container. Thus in the cutting state, the cutting element and/or tissue engagement component can cut or penetrate the skin surface and underlying tissue of a patient. The locking mechanism may allow the plunger, tissue engagement component and/or cutting element may be unlocked from the cutting state so that they can be retracted into the container and/or plunger sleeve. In other embodiments there may be no plunger sleeve and the plunger may directly contact inner walls of the container. In some examples, the plunger sleeve may be an integral part of the container and in other examples the plunger sleeve may be a separate component which may be fitted inside the container and/or fixed to inner walls of the container.

In some embodiments, the first sleeve connector member is configured to be moved between: a first position where the first sleeve connector member is configured to abut an end of the first plunger to inhibit movement of the first plunger with respect to the plunger sleeve; and a second position where the first sleeve connector member is configured to enable movement of the first plunger with respect to the plunger sleeve.

In some embodiments, the second sleeve connector member is configured to be moved between: a first position where the second sleeve connector member is configured to abut the container rib inside the container body to inhibit movement of the sleeve and the first plunger with respect to the container body; a second position where the second sleeve connector member is configured to abut the step to inhibit movement of the second plunger with respect to the plunger sleeve; and a third position where the second sleeve connector member is configured to enable movement of the second plunger with respect to the plunger sleeve.

In some embodiments, the first sleeve connector member is integrally formed with the plunger sleeve and is bendable; and the second sleeve connector member is integrally formed with the plunger sleeve and is bendable.

In some embodiments, the first sleeve connector member comprises a first projection, the first projection being configured to abut an end of the first plunger and the second plunger; and the second sleeve connector member comprises a second projection, the second projection being configured to abut the step of the second plunger.

In some embodiments, the second sleeve connector member is biased towards the first position such that longitudinal movement of the intermediate portion of the second plunger away from the first container outlet causes the second sleeve connector member to move to the first position.

In some embodiments, the second plunger comprises a plunger locking system configured to lock the second plunger in the retracted position.

In some embodiments, the plunger locking system comprises a first elongate locking member and a second elongate locking member, and the first elongate locking member and the second elongate locking member are configured to abut the container rim when the second plunger is in the retracted position, thereby locking the second plunger in the retracted position.

In some embodiments, the tissue retrieval device further comprises a plunger latch system comprising: a plunger sleeve; a first latch member that is pivotably mounted to the plunger sleeve; and a second latch member that is pivotably mounted to the plunger sleeve.

In some embodiments, the first latch member is pivotable between: a first position where the first latch member is configured to abut the container rim to inhibit movement of the plunger sleeve through the inner container hole; and a second position where the first latch member is configured to enable movement of the plunger sleeve through the inner container hole.

In some embodiments, the second plunger comprises a first latch member projection that is configured to contact the first latch member at an actuation end of the first latch member to pivot the first latch member from the first position to the second position with longitudinal movement of the second plunger towards the plunger.

In some embodiments, the second latch member is pivotable between: a first position where the second latch member is configured to abut the plunger to inhibit movement of the plunger with respect to the plunger sleeve; and a second position where the second latch member is configured to enable movement of the plunger with respect to the plunger sleeve.

In some embodiments, the plunger sleeve is configured to abut the container rim with longitudinal movement of the plunger sleeve towards the distal volume.

In some embodiments, the tissue retrieval device further comprises a plunger sleeve, wherein: the plunger sleeve comprises a plunger sleeve rim that extends inwardly in a radial direction of the plunger sleeve to define an inner plunger sleeve hole; and the plunger sleeve defines: a first plunger sleeve opening; and a plunger sleeve containment volume between the first plunger sleeve opening and the inner plunger sleeve hole; wherein the plunger sleeve is configured to contain at least part of the plunger within the plunger sleeve containment volume.

In some embodiments, the cutting element is mounted to the plunger sleeve.

In some embodiments, the plunger is rotatable with respect to the plunger sleeve and/or container.

In some embodiments, the plunger comprises a plunger keying feature; and the plunger is rotatable from a first position where the plunger keying feature is configured to abut the plunger sleeve rim to inhibit movement of the plunger through the inner plunger sleeve hole to a second position where the plunger keying feature is configured to enable movement of the plunger through the inner plunger sleeve hole.

In some embodiments, the plunger keying feature further comprises an elongate locking member that is configured to abut the plunger sleeve rim when the plunger is in a first retracted position, thereby inhibiting movement of the plunger with respect to the plunger sleeve.

In some embodiments, the tissue engagement component is contained within the plunger sleeve volume when the first plunger is in the first retracted position.

In some embodiments, the plunger sleeve comprises an elongate plunger sleeve locking member that is configured to abut the plunger sleeve rim when the plunger is in a second retracted position, thereby inhibiting movement of the plunger sleeve with respect to the container.

In some embodiments, the plunger comprises a sealing component such as, but not limited to, an O-ring. In some examples the plunger comprises an O-ring and an O-ring groove configured to receive the O ring.

In some embodiments, the O-ring is configured to seal with an inner surface of the plunger sleeve.

In some embodiments, the plunger comprises a plunger keying feature that is configured to abut the container rim to inhibit movement of the plunger through the inner container hole.

In some embodiments, the plunger is rotatable from a first position where the plunger keying feature is configured to abut the container rim to inhibit movement of the plunger through the inner container hole to a second position where the plunger keying feature is configured to enable movement of the plunger through the inner container hole.

In some embodiments, the plunger keying feature comprises an elongate locking member that is configured to abut the container rim when the plunger is in a retracted position, thereby inhibiting movement of the plunger with respect to the container.

In some embodiments, the plunger comprises an O-ring and an O-ring groove configured to receive the O ring.

In some embodiments, the O-ring is configured to seal with an inner surface of the container.

In some embodiments, the cutting element is mounted to the container.

In some embodiments, the plunger comprises a frangible portion at which the plunger is configured to be broken.

In some embodiments, the second plunger comprises a frangible portion at which the second plunger is configured to be broken.

In some embodiments, the tissue retrieval device further comprises a locking element that is configured to engage with the plunger to inhibit movement of the plunger.

In some embodiments, the tissue retrieval device further comprises a locking element that is configured to engage with the second plunger to inhibit movement of the second plunger.

According to a second aspect of the present disclosure, there is provided a device for treating a subcutaneous skin abscess. The device may be referred to as a tissue retrieval device may comprise: a plunger comprising a plunger sealing surface; a container configured to contain at least part of the plunger; wherein the plunger sealing surface is configured to seal with an inner surface of the container to inhibit fluid flow between the plunger sealing surface and the inner surface of the container; and a cutting element connected to the container, wherein: the cutting element is configured to cut tissue of a patient; and the cutting element defines a cutting element opening; wherein the plunger is configured to be moved away from the cutting element to generate a suction pressure at the cutting element opening.

In some embodiments, the tissue retrieval device further comprises a pair of opposed stabilising members, wherein an outer surface of each of the par of opposed stabilising members is configured to contact a patient's skin to stabilise the tissue retrieval device during use.

In some embodiments, the container comprises the stabilising members.

In some embodiments, the stabilising members extend away from a central axis of the container in a radial direction.

In some embodiments, the plunger comprises an elastomeric sealing portion.

In some embodiments, the plunger comprises an O-ring and an O-ring groove configured to receive the O ring.

In some embodiments, the O-ring comprises the plunger sealing surface.

In some embodiments, the plunger comprises a frangible portion at which the plunger is configured to be broken.

In some embodiments, the cutting element comprises a cylindrical cutting sharp extending from a cutting end portion to a second end portion to define a cutting lumen.

In some embodiments, there is provided a device comprising: a tissue engagement component comprising a tapered projection; a container configured to contain at least part of the tissue engagement component; and a cutting element configured to cut tissue of a patient; wherein the tissue engagement component is configured to engage the tissue cut by the cutting element such that movement of the tissue engagement component into a containment volume of the container causes movement of at least part of the tissue cut by the cutting element into the containment volume.

In some embodiments, the tapered projection is configured to engage the tissue cut by the cutting element such that movement of the tissue engagement component into the containment volume of the container causes movement of at least part of the tissue cut by the cutting element into the containment volume.

In some embodiments, the tapered projection tapers from a base to a point.

In some embodiments, the tissue engagement component defines a tissue engagement component lumen that extends between a first tissue engagement component opening and a second tissue engagement component opening; and the tapered projection projects into the tissue engagement component lumen.

In some embodiments, the tissue engagement component defines a tissue engagement opening that is fluidly connected to the tissue engagement component lumen.

In some embodiments, the tissue retrieval device comprises a plurality of tapered projections and a plurality of tissue engagement openings.

In some embodiments, the tissue retrieval device further comprises a plunger configured to move in a longitudinal direction of the container, wherein: the container is configured to contain at least part of the plunger; and movement of the plunger causes movement of the tissue engagement component.

In some embodiments, the tissue retrieval device further comprises a plunger interface component, wherein: the tissue engagement component is connected to a first end portion of the plunger interface component; and the plunger is connected to a second end portion of the plunger interface component.

In some embodiments, the plunger is configured to be moved between: a first position where at least part of the tissue engagement component is disposed within a volume defined by the cutting element; and a second position where the tissue engagement component is contained within the containment volume of the container.

In some embodiments, the cutting element comprises a cylindrical cutting sharp extending from a cutting end portion to a second end portion to define a cutting lumen.

In some embodiments, the container defines the containment volume and a distal volume; the container comprises a container rim that extends inwardly in a radial direction of the container to define an inner container hole; the container rim is between the containment volume and the distal volume; and the inner container hole fluidly connects the containment volume and the distal volume.

In some embodiments, the cutting element is mounted to the container.

In some embodiments, the plunger comprises an O-ring and an O-ring groove configured to receive the O-ring.

In some embodiments, the O-ring is configured to seal with an inner surface of the container.

In some embodiments, the plunger comprises a frangible portion at which the plunger is configured to be broken.

According to a third aspect of the present disclosure, there is provided a percutaneous drainage device for draining a subcutaneous skin abscess. The percutaneous drainage device may comprise: a cannula defining a cannula lumen that extends between a first cannula opening and a second cannula opening; and a penetration component configured to be slidably received within the cannula, the penetration component comprising: a penetration portion configured to penetrate tissue of a patient, thereby enabling introduction of the first cannula opening to a subcutaneous fluid collection site; and a penetration component lumen that extends between a first penetration component opening and a second penetration component opening, the penetration component lumen being configured to receive a guide wire.

In some embodiments, the cannula comprises a recess; and the penetration component comprises a projecting feature; wherein the recess is configured to receive the projecting feature when the penetration component is received within the cannula, thereby impeding rotation of the cannula with respect to the penetration component.

In some embodiments, the penetration portion comprises a penetration portion that is tapered towards a penetrating end of the penetration component.

In some embodiments, the penetration portion is configured to: extend through the first cannula opening; and be driven into the tissue of the patient by rotation of the penetration component, thereby driving the cannula into the tissue of the patient.

In some embodiments, the percutaneous drainage device further comprises a cap defining a cap lumen that extends from a first cap opening to a second cap opening, the cap lumen being configured to receive the cannula and the penetration component.

In some embodiments, the cap lumen comprises: a first tapered portion that extends from the first cap opening towards the second cap opening, wherein a cross sectional area of the first tapered portion decreases as the first tapered portion extends from the first cap opening towards the second cap opening; a second tapered portion, wherein a cross sectional area of the second tapered portion increases as the second tapered portion extends away from the first cap opening towards the second cap opening; and a third tapered portion that extends from the second tapered portion towards the second cap opening, wherein a cross sectional area of the third tapered portion increases as the third tapered portion extends away from the second tapered portion towards the second cap opening.

In some embodiments, the cap lumen comprises a guide portion extending between the first tapered portion and the second tapered portion.

In some embodiments, the first penetration component opening is aligned with the guide portion of the cap lumen when the penetration component is received in the cap.

In some embodiments, the cap comprises a cap groove that extends from the first cap opening to the second cap opening, the cap groove being configured to enable the passage of guide wire from the cap lumen to outside of the cap.

In some embodiments, the penetration component comprises a gripping portion, the gripping portion defining the second penetration component opening.

In some embodiments, the percutaneous drainage device further comprises a penetration component cap configured to removably engage with the penetration component to enable selective obstruction of the second penetration component opening.

In some embodiments, the cannula defines a cannula thread.

In some embodiments, the percutaneous drainage device further comprises a collar configured to: removably engage with the cannula; and be movable in a longitudinal direction of the cannula.

In some embodiments, the percutaneous drainage device further comprises a connecting component comprising: a collar engaging portion configured to removably engage with the collar; and an extension portion extending radially from the collar contacting portion, the extension portion being configured to connect to the tissue of the patient.

According to a fourth aspect of the present disclosure, there is provided a percutaneous drainage device for draining a subcutaneous skin abscess. The percutaneous drainage device may comprise: a cannula defining a cannula lumen that extends between a first cannula opening and a second cannula opening; a penetration component configured to be slidably received within the cannula lumen, the penetration component comprising a penetration portion configured to penetrate tissue of a patient, thereby enabling introduction of the first cannula opening to a subcutaneous fluid collection site; a collar configured to: removably engage with the cannula; and be moveable in a longitudinal direction of the cannula; and a connecting component comprising: a collar engaging portion configured to removably engage with the collar; and an extension portion extending radially from the collar engaging portion, the extension portion being configured to connect to the tissue of the patient.

In some embodiments, the collar is configured to be movable in the longitudinal direction of the cannula while engaged with the cannula.

In some embodiments, the cannula defines a cannula thread; and the collar comprises a collar lumen configured to receive the cannula and to cooperate with the cannula thread such that: rotation of the collar with respect to the cannula causes movement of the collar in the longitudinal direction of the cannula; and movement of the collar in the longitudinal direction of the cannula is impeded without rotation of the collar with respect to the cannula.

In some embodiments, the collar defines a peripheral hole.

In some embodiments, the collar defines a channel between an inner channel wall and an outer channel wall.

In some embodiments, the channel is an annular channel that extends around at least a portion of the cannula lumen.

In some embodiments, the cannula lumen extends between a first collar opening and a second collar opening; the inner channel wall extends further away from the first collar opening than the outer channel wall; and the inner channel wall is configured to contact the cannula.

In some embodiments, the cannula defines a cannula flange; and the inner channel wall is configured to contact the cannula thread and the cannula flange.

In some embodiments, the collar engaging portion defines a collar engaging portion opening that is configured to enable passage of the penetration component and the cannula.

In some embodiments, the collar engaging portion comprises a collar engaging projection that is configured to be received by the collar to inhibit relative movement between the collar and the collar engaging portion.

In some embodiments, the collar engaging projection is an annular projection that is configured to be received by the channel of the collar.

In some embodiments, the collar engaging portion comprises a locating projection that is configured to be received by the peripheral hole to inhibit rotation of the collar engaging portion with respect to the collar.

In some embodiments, the extension portion comprises an adhesive portion configured to adhere to skin of the patient.

The features of the first and second aspects of the present disclosure may be freely combined or used together. Features of the third and fourth aspects of the disclosure may be freely combined or used together. A fifth aspect of the present disclosure provides a system or kit of parts for treating a subcutaneous skin abscess comprising a first device according to the first or second aspect, together with a second device according to the third or fourth aspect. The system or kit of parts may further include a guide wire for guiding the second device to the subcutaneous skin abscess.

A sixth aspect of the present disclosure provides a device for removing tissue above a subcutaneous skin abscess, the device comprising: a cutting element configured to cut through the skin surface of a patient to cut out a skin plug from epidermal and dermal tissue of a patient; a tissue engagement component for engaging the skin plug; and a container configured to contain at least part of the tissue engagement component; wherein the tissue engagement component is movable relative to the container and configured to engage the skin plug cut by the cutting element such that movement of the tissue engagement component into a containment volume of the container causes movement of at least part of the skin plug cut by the cutting element into the containment volume.

The tissue engagement component may be harpoon shaped. In some embodiments, the tissue engagement component comprises a penetration portion, a barb and a shaft. The penetration portion may be at a distal end of the shaft and configured for penetrating the skin and tissue of a patient, the barb may be positioned on the shaft behind the penetrating portion and configured for hooking tissue of the patient to pull on and retrieve a skin plug cut by the cutting element when the tissue engagement component is retracted or moved into the container.

In other embodiments, the tissue engagement component may comprise a pair of jaws. The jaws may have a skin piercing distal end and jaw teeth for grabbing the tissue.

In other embodiments, the tissue engagement portion may comprise a cylindrical component defining a lumen between a distal end and proximal end of the cylindrical component, wherein the cylindrical component comprises a plurality of apertures and a tapered projection associated with each aperture, the tapered projection projecting radially inward and towards a proximal end of the tissue engagement component. The tapered projections may act to snare and engage with a skin plug to pull the skin plug back into the container when the tissue engagement component is retracted.

The sixth aspect may be used any of the features of the first to fifth aspects described above, except where the features are mutually exclusive.

### BRIEF DESCRIPTION OF THE DRAWINGS

Embodiments of the disclosure are now described, by way of example only, with reference to the accompanying drawings, in which:

FIG. 1A is a schematic drawing of a shallow subcutaneous skin abscess;

FIG. 1B is a schematic drawings of a deep subcutaneous skin abscess;

FIG. 1C shows a device used to cut a skin plug in a first stage of draining a shallow subcutaneous skin abscess according to some embodiments;

FIG. 1D shows a second stage of draining a shallow subcutaneous skin abscess in which the device of FIG. 1C is used to drain pus from the abscess according to some embodiments;

FIG. 1E is a cross-sectional view showing cutting of a skin plug by the device of FIG. 1C according to some embodiments;

FIG. 1F shows a first stage of draining a deep subcutaneous skin abscess in which a first device is used to cut a skin plug in according to some embodiments;

FIG. 1G shows a second stage of draining a deep subcutaneous skin abscess in which a second device is guided by a guide wire towards the abscess according to some embodiments;

FIG. 1H shows a third stage of draining a deep subcutaneous skin abscess in which a cannula of the second device is secured to the skin surface with a collar according to some embodiments;

FIG. 1I shows a fourth stage of draining a deep subcutaneous skin abscess in which a retraining device is attached to the collar to further secure the cannula of the second device according to some embodiments;

FIG. 2A shows a perspective view of a tissue retrieval device, according to some embodiments;

FIG. 2B shows an exploded view of the tissue retrieval device of FIG. 2A according to some embodiments;

FIG. 2C shows a perspective view of a container body of the tissue retrieval device of FIG. 2A, with the container body being rendered as transparent, according to some embodiments;

FIG. 3 shows a cross-sectional view of a container of the tissue retrieval device of FIG. 2A, the cross-sectional view corresponding to a side view of the container, according to some embodiments;

FIG. 4A shows a top view of the tissue retrieval device of FIG. 2A, in a first state, indicating a section plane, according to some embodiments;

FIG. 4B shows a side view of the tissue retrieval device of FIG. 2A, in the first state, when sectioned in accordance with the section plane of FIG. 4A, according to some embodiments;

FIG. 5A shows a side view of the tissue retrieval device of FIG. 2A, in the first state, indicating a second section plane, according to some embodiments;

FIG. 5B shows a top view of the tissue retrieval device of FIG. 2A, in the first state, when sectioned in accordance with the second section plane of FIG. 5A, according to some embodiments;

FIG. 6A shows a top view of the tissue retrieval device of FIG. 2A, in a second state, indicating a section plane, according to some embodiments;

FIG. 6B shows a side view of the tissue retrieval device of FIG. 2A, in the second state, when sectioned in accordance with the section plane of FIG. 6A, according to some embodiments;

FIG. 7A shows a side view of the tissue retrieval device of FIG. 2A, in the second state, indicating a second section plane, according to some embodiments;

FIG. 7B shows a bottom view of the tissue retrieval device of FIG. 2A, in the second state, when sectioned in accordance with the section plane of FIG. 7A, according to some embodiments;

FIG. 8A shows a top view of the tissue retrieval device of FIG. 2A, in a third state, indicating a section plane, according to some embodiments;

FIG. 8B shows a side view of the tissue retrieval device of FIG. 2A, in the third state, when sectioned in accordance with the section plane of FIG. 8A, according to some embodiments;

FIG. 9A shows another side view of the device of FIG. 2A, with the tissue retrieval device being in the third state, indicating a section plane, according to some embodiments;

FIG. 9B shows a bottom view of the tissue retrieval device of FIG. 2A, in the third state, when sectioned in accordance with the section plane of FIG. 9A, according to some embodiments;

FIG. 10A shows a top view of a part of the tissue retrieval device of FIG. 2A, in a fourth state, indicating a section plane, according to some embodiments;

FIG. 10B shows a side view of the part of the tissue retrieval device shown in FIG. 10A, when sectioned in accordance with the section plane of FIG. 10A, according to some embodiments;

FIG. 11A shows a top view of a second part of the tissue retrieval device of FIG. 2A, in the fourth state, indicating a second section plane, according to some embodiments;

FIG. 11B shows a side view of the second part of the tissue retrieval device shown in FIG. 11A, when sectioned in accordance with the second section plane of FIG. 11A, according to some embodiments;

FIG. 12A shows a perspective view of another tissue retrieval device, according to some embodiments;

FIG. 12B shows a bottom view of the tissue retrieval device of FIG. 12A, according to some embodiments;

FIG. 12C shows a side view of the tissue retrieval device of FIG. 12A, according to some embodiments;

FIG. 12D shows a perspective view of a portion of the tissue retrieval device of FIG. 12A, in a fourth state, according to some embodiments;

FIG. 13 shows an exploded view of the tissue retrieval device of FIG. 12A, according to some embodiments;

FIG. 14A shows a side view of the tissue retrieval device of FIG. 12A, showing a section plane of the tissue retrieval device in a first state, according to some embodiments;

FIG. 14B shows a side view of the tissue retrieval device of FIG. 12A, showing a section plane of the tissue retrieval device in a second state, according to some embodiments;

FIG. 14C shows a side view of the tissue retrieval device of FIG. 12A, showing a section plane of the tissue retrieval device in a third state, according to some embodiments;

FIG. 15A shows a side view of a part of the tissue retrieval device of FIG. 12A, showing a section plane of the part of tissue retrieval device in the third state, according to some embodiments;

FIG. 15B shows a side view of a part of the tissue retrieval device of FIG. 12A, showing a section plane of the part of tissue retrieval device in a fourth state, according to some embodiments;

FIG. 16A shows a perspective view of another tissue retrieval device, according to some embodiments;

FIG. 16B shows an exploded view of the tissue retrieval device of FIG. 16A, according to some embodiments;

FIG. 17A shows a top view and a rear view of the tissue retrieval device of FIG. 16A, according to some embodiments;

FIG. 17B shows a side view and a rear view of the tissue retrieval device of FIG. 16A, according to some embodiments;

FIG. 17C shows another perspective view of the tissue retrieval device of FIG. 16A, according to some embodiments;

FIG. 18A shows a side view of the tissue retrieval device of FIG. 16A, showing a section plane of the tissue retrieval device in a first state, according to some embodiments;

FIG. 18B shows a top view of the tissue retrieval device of FIG. 16A, showing a section plane of the tissue retrieval device in the first state, according to some embodiments;

FIG. 19 shows an exploded view of a percutaneous drainage device, according to some embodiments;

FIG. 20 shows a perspective view of a cannula, a penetration component and a collar of the percutaneous drainage device of FIG. 19, according to some embodiments;

FIG. 21 shows a sequence of steps of use of the percutaneous drainage device of FIG. 19, according to some embodiments;

FIG. 22 shows a perspective view and a top view of the percutaneous drainage device of FIG. 19, according to some embodiments;

FIG. 23 shows a first side view and a second side view of the percutaneous drainage device of FIG. 19, according to some embodiments; and

FIGS. 24A to 24F show another example of a device for removing tissue above a subcutaneous skin abscess and/or draining a subcutaneous skin abscess.

### DESCRIPTION OF EMBODIMENTS

Part 1: Overview of Devices and Systems for Removing Tissue Above and/or Draining a Subcutaneous Skin Abscess

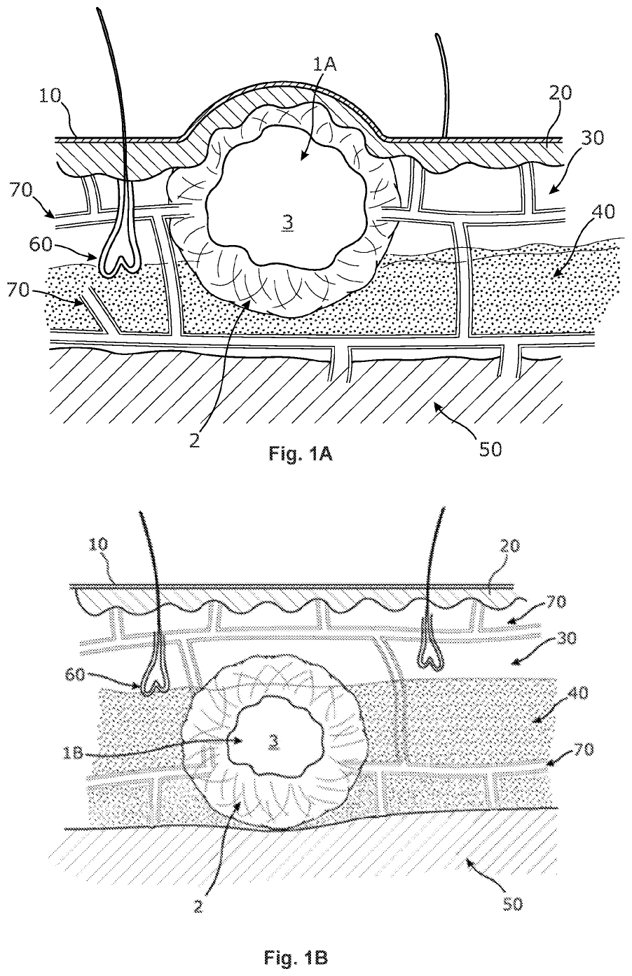

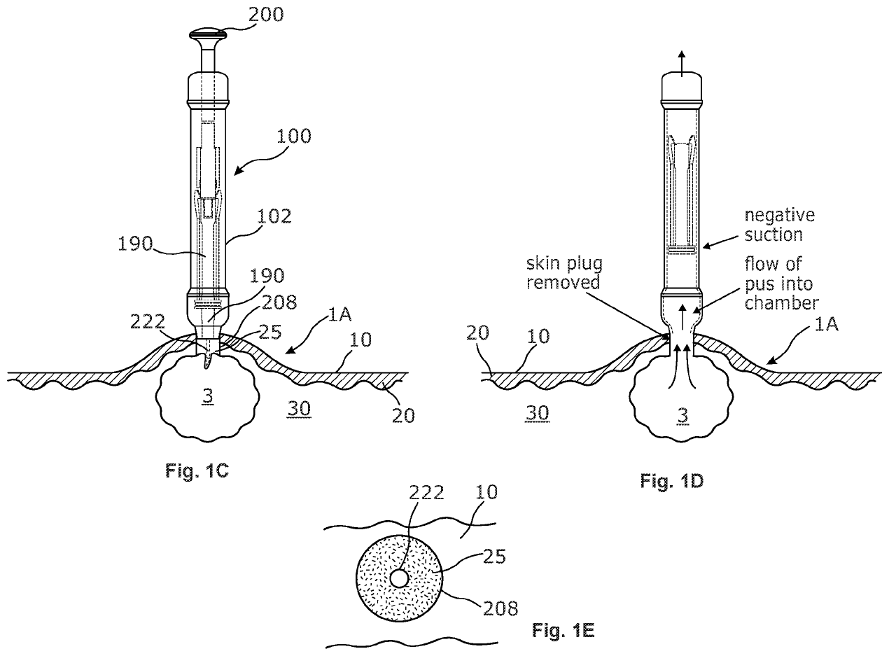

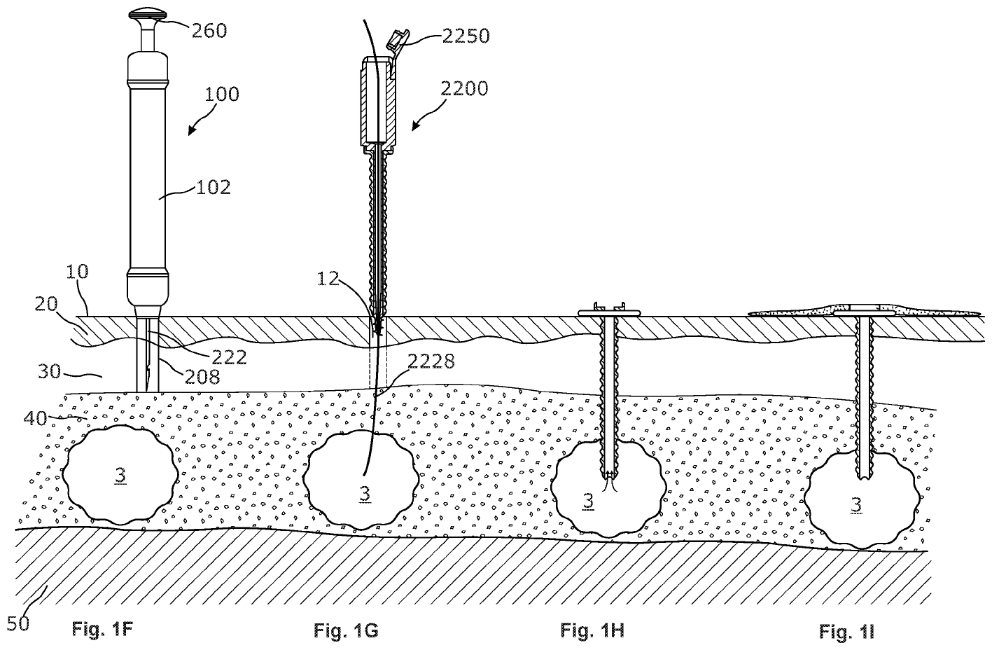

FIGS. 1A and 1B show examples of subcutaneous skin abscesses in the layers below the surface of the skin. FIG. 1A shows a shallow skin abscess 1A, which generally occurs in the dermis, while FIG. 1B shows a deep skin abscess 1B, which may form between the dermis and underlying muscle in the subcutaneous fat layer. The abscess is typically filled with pus 3, which may contain a mixture of liquid and solid matter. In order to prevent further infection, it is desirable to drain the abscess.

FIGS. 1A and 1B show that below the surface of the skin 10, there is an epidermis layer 20, dermis layer 30, subcutaneous fat layer 40 and muscle layer 50. The epidermis 20 is a layer is directly below the skin surface and typically has a thickness of up to 1 mm, while the dermis layer typically extends a further 1 mm to 4 mm below the skin surface. In the context of this disclosure, a superficial skin abscess means an abscess having a depth up to 2 cm or so below the skin surface and which occurs within the dermis layer 30 and subcutaneous fat layer 40, but not further, e.g. as shown in FIG. 1A. A deep skin abscess means an abscess which has a depth of greater than 2 cm below the skin surface and extends into the subcutaneous fat layer 40, e.g. as shown in FIG. 1B. Subcutaneous skin abscesses are distinguished from abscesses in internal organs, such as in the peritoneal cavity (which contains the intestines, stomach and liver), in that subcutaneous skin abscesses do not extend beyond the subcutaneous fat layer into muscle or other tissue below. For example, a subcutaneous skin abscess is entirely above and does not extend into the muscle layer 50. Cysts and abscesses in internal body organs are always below the subcutaneous fat layer and often below a layer of muscle too.

As shown in FIGS. 1A and 1B, blood vessels 70 may extend through the subcutaneous fat layer 40 and hair follicles 60 and associated hairs may extend from the subcutaneous fat layer up to the surface of the skin. There may be swollen tissue 2 surrounding the pus 3 of the skin abscess, wherein the swollen tissue 2 is formed of the tissue of the surrounding layer(s) such as dermis and/or subcutaneous fat.

The present disclosure is concerned with devices for draining subcutaneous skin abscesses. In some examples the devices are suitable for draining subcutaneous skin abscesses which extend up to 6 cm below the surface of the skin.

Skin abscesses that are extensively large, deep, or which have thick or non homogenous contents can be challenging to drain and achieve clinical resolution of the abscess. Some skin abscesses can be drained percutaneously by inserting a needle, catheter or other suitable drain through the skin into the abscess to remove or drain the fluid collection; however, such drains may become occluded with abscess contents and stop flowing before the entire contents have been cleared. The patient then requires additional treatment, possibly including incision and drainage surgery to open the abscess using an incision. Some of the devices disclosed in this application facilitate access to a subcutaneous skin abscess by removing the skin layers above abscess and/or facilitating entry of a cannula to the abscess in a safe manner which does not require in-patient surgery and/or using suction to induce the flow of the abscess contents into a container or cannula.

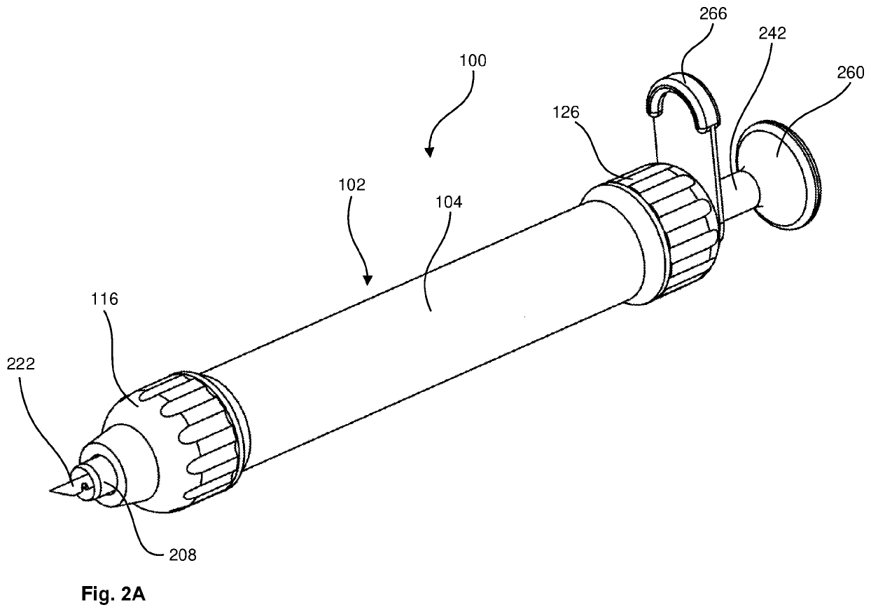

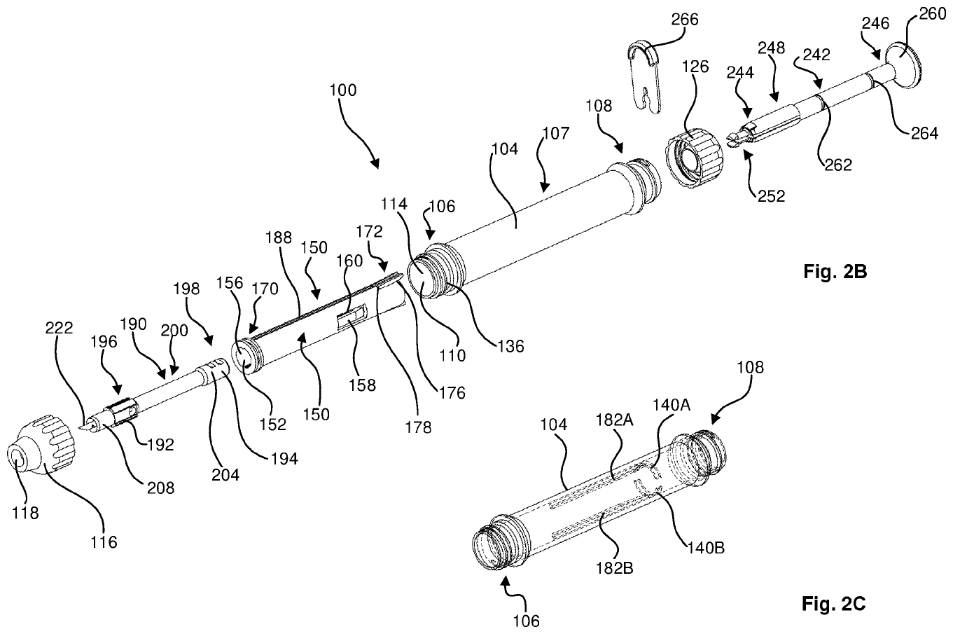

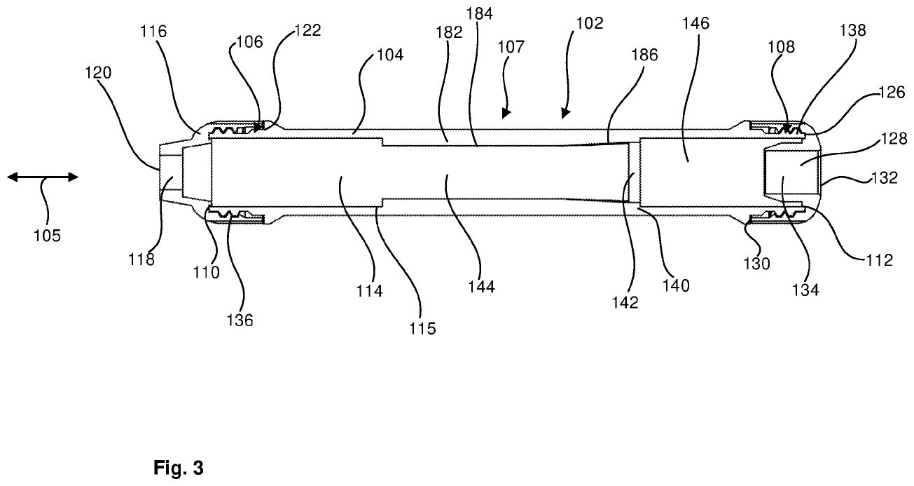

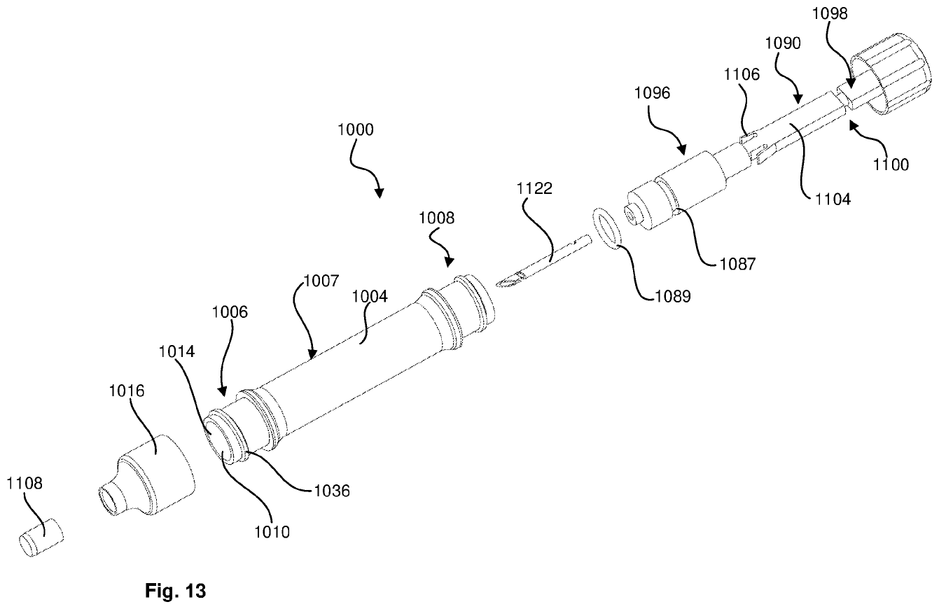

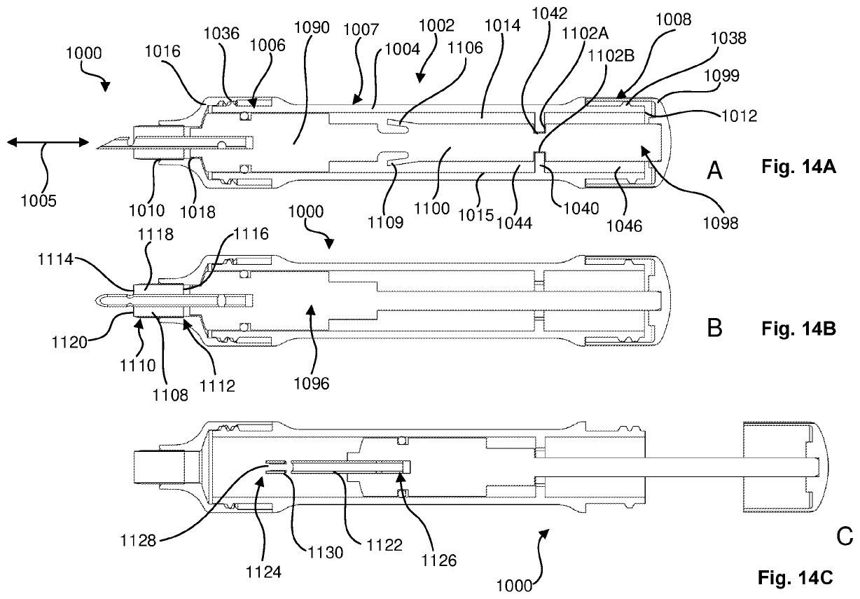

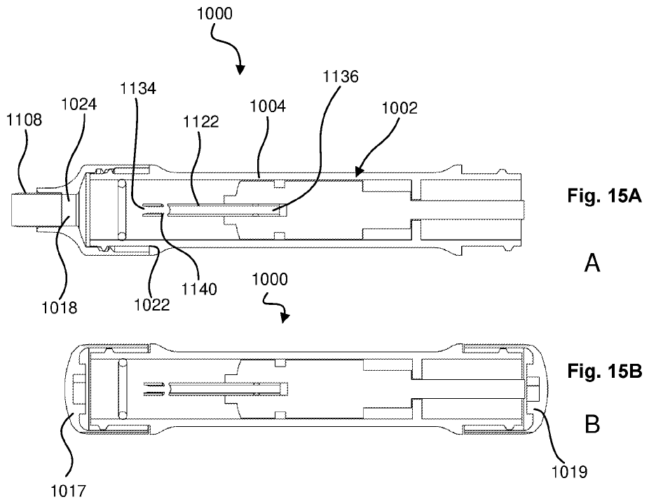

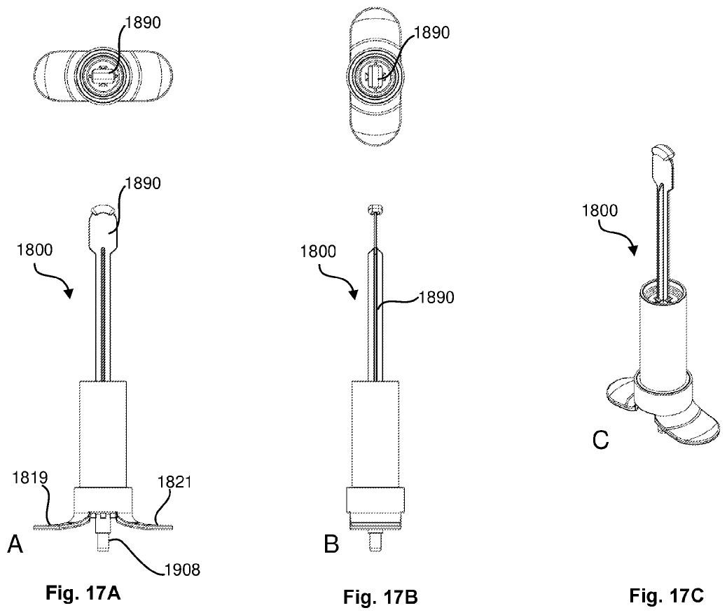

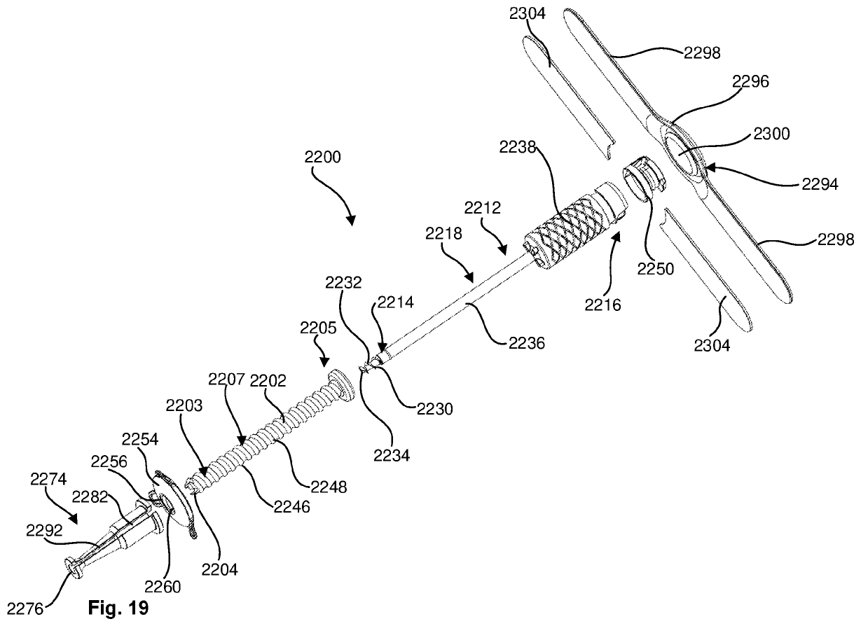

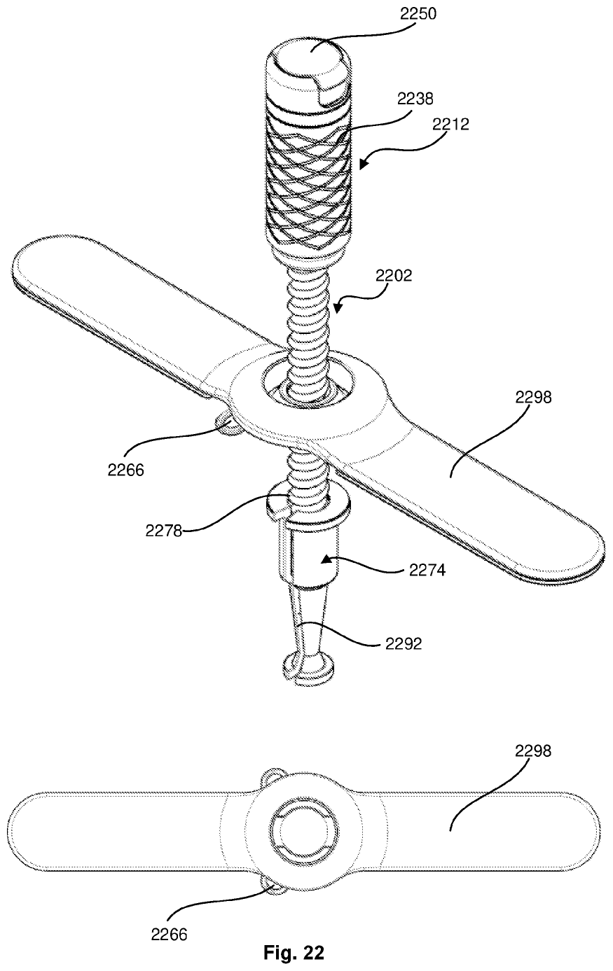

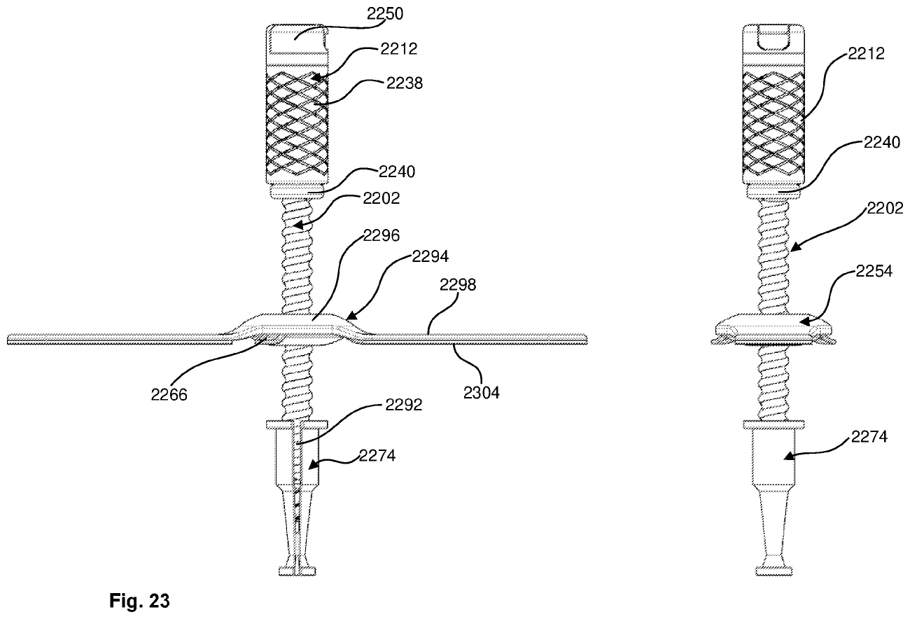

The present disclosure discusses two different types of percutaneous drainage device for draining a subcutaneous skin abscess. An example of a first type of percutaneous drainage device is shown in FIGS. 2A-11, FIGS. 12-15 and FIGS. 16A to 18B. An example of the second type of percutaneous drainage device is shown in FIGS. 19-23.

The first type of percutaneous drainage device 100 comprises a container 102, a tissue engagement component 222 and a cutting element 208. In some examples the tissue engagement component 222 is in the form of a harpoon. The cutting element 208 may be curved and in some examples is in the form of a cylindrical blade. In some examples, the cutting element 208 defines a lumen which surrounds at least a portion of the tissue engagement component 222. The cutting element 208 extends outwardly from a distal end of the container and may be used to cut out a section of the skin and underlying layers to form a ‘skin plug’. The tissue engagement portion 222 may be used to penetrate and attach to the skin plug and remove skin plug when the tissue engagement portion is retracted into the container. The first type of percutaneous drainage device may be used to remove a skin plug. Removing the skin plug may allow the first device to directly access a shallow skin abscess, or may provide an opening through which the second type of device can reach a deeper abscess.

FIG. 1C shows how the first type of percutaneous drainage device may be used to drain a shallow abscess. The shallow skin abscess may have developed in the dermis and may be visible at the skin surface (referred to as a ‘pointing’ abscess). The cutting element 208 is used to cut a section of the skin, for instance by placing the blade (cutting element) on the skin surface and rotating while pushing downwards the cutting element may create a defect in the skin layers, which may take the form of a circular cut in the skin surface, epidermis and dermis. As the cutting element may cut by being rotated, rather than pushing only as in the manner of a scalpel blade, this may cause less discomfort and also be safer as it is less likely to cut accidentally cut through body parts which are not intended to be cut. The cutting element may form a blade with a cutting edge at the distal end. The distal end of the cutting element be tapered or bevelled to form a sharp cutting edge. However, the cutting edge may present a level or flat cutting plane. For instance, when the device is held with container 102 perpendicular to the skin surface, the cutting edge of the cutting element may be substantially level with respect to the skin. For example, as shown in FIG. 2A, the cutting element 208 has a cutting edge which forms a level plane, substantially perpendicular to the longitudinal axis of the container. This is in contrast to the tissue retrieval component 222 which has an angled cutting edge more similar to a scalpel. In the example of FIG. 2A, the cutting element 208 is cylindrical and defines a lumen, such that the cutting edge forms a circle which contacts the skin when the device is used to cut the skin surface. The level or flat cutting plane improves safety and allows consistency and predictability in the depth of skin which is cut.

The section of skin, epidermis and dermis inside the cut may be referred to as a ‘skin plug’. The tissue engagement portion 222 may penetrate the skin plug and a barb of the tissue engagement portion may engage with or attach to the skin plug. The cutting element and tissue engagement portion may be pushed deep enough into the skin to contact the skin abscess which is to be drained. Then as shown in FIG. 1D, the tissue engagement portion 222 may be retracted into the container 102. When retracted the tissue engagement portion 222 takes the skin plug with it and leaves behind an opening through which the contents, e.g. pus 3, of the skin abscess may flow into the container. In this way the shallow subcutaneous skin abscess may be drained. FIG. 1E is a cross-sectional view showing an example of a portion of skin 10 which has been cut by the device 100. The cutting element 208 may form a circular cut defining a volume of tissue or ‘skin plug’ 25 inside and the tissue engagement component 222 engages with and grabs hold of the skin plug 25 so that it may be removed by retracting the tissue engagement component. It will be appreciated from the above, that the first device is configured to first cut a volume of tissue (the ‘skin plug’) with the cutting element so that the skin plug is detached from surrounding tissue and then extract the skin plug with the tissue engagement component. This is different to other devices, such as morcellators, which first grab tissue (often through an existing hole cut by a knife) and then sever the connection of the grabbed tissue to the body after the tissue and grabbing element have been withdrawn.

In some examples, the cutting element 208 may have a length of 5-10 mm; in other examples 5-8 mm, in some examples of about 7 mm. This is envisaged to be sufficiently long to cut into the dermis and create a skin plug, but not so long as to extend deep into subcutaneous fat. In some examples, the cutting element 208 may have an outer diameter of between 3 mm and 6 mm, in some examples 4 mm-6 mm, in some examples 3 mm-5.5 mm, in other examples around 4.5 mm-5.5 mm and in some examples about 5 mm. The diameter of the cutting element determines the size of the skin plug. The cutting element diameter should be sufficient to create a large enough skin plug to provide access and/or facilitate subcutaneous skin abscess drainage. Thus, especially where there is significant viscous fluid to drain the diameter of the cutting element may be 3 mm or greater. Where the diameter is larger than 6 mm, scarring of tissue becomes a concern.

The diameter of the cutting element should be sufficient to allow the tissue engagement component to withdraw a skin plug of larger diameter than the tissue engagement component through the cutting element lumen. In some examples the tissue engagement component may have a diameter of 1 mm to 3 mm. The tissue engagement component may extend within a lumen of the cutting element; in some examples, the clearance between the shaft of the tissue engagement component and the inner diameter of the cutting element may be 1 mm-3 mm, in some examples 2 mm-3 mm, in still other examples 2 mm-2.5 mm.

In example of FIGS. 1C-1D, both the cutting element 208 and the tissue engagement component 222 are retractable into the container 102. For example the cutting element and the tissue engagement component may be attached or mechanically linked to a plunger 190 which is received in the container. In that case the plunger may be movable towards the distal end of the container so that the tissue engagement component and cutting element protrude from an opening in the distal end of the container and may be movable towards the proximal end of the container so that the tissue engagement component and cutting element are retracted into the container. The device may be configured to generate a negative pressure or suction when the tissue engagement component is retracted into the container. This may help start the flow of pus and/or other contents of the skin abscess into the container. As the pus in a skin abscess may include a mixture of solid and liquid and may be relatively viscous, the suction may be needed to start the flow of pus out of the abscess and into the container.

In some examples, the plunger, or a sealing component (such as an o-ring) connected to the plunger, may seal with an inner part of the container so that a negative pressure or vacuum is generated by withdrawal of the plunger towards the proximal end of the container when the tissue engagement element is retracted. The plunger or sealing component may seal with the inner walls of the container, with a sleeve received in the container or with another internal part of the chamber which forms a chamber for receiving the contents of the abscess. The plunger may be comprise one or more parts and may be movable by a handle 260. The cutting element 208 and the tissue engagement component 222 may be configured to adopt a plurality of states including a cutting state and a retractable state. In the cutting state the cutting element and tissue engagement component may be fixed relative to the container, e.g. locked in place, in a position in which they extend from a distal end of the container. When in the cutting state the cutting element and tissue engagement component may be used to cut the skin, epidermis and dermis layers as they do not retract when pushed against and/or into the skin. In the retractable state the cutting element and tissue engagement component may be moved relative to the container to retract into the container.

In some examples, the tissue engagement component and the cutting element may be completely removed from the container. This may be conveniently achieved by retracting the plunger, tissue engagement component and cutting element all the way through the container and out through an opening in the proximal end of the container. The two ends of the container may then be closed, e.g. by end caps. This enables the container to store the retrieved tissue and/or abscess contents to be sent for analysis. In such cases it is desirable to remove the cutting element and tissue engagement component so no sharp blades or edges or left in the sample.

In other examples, the tissue engagement component and cutting element may be retractable, but not removed, which may be sufficient if the device is not used for pathology to provide a sample for analysis. In some examples the tissue engagement component may be retractable, but the cutting element may be non-retractable. For instance, the cutting element may be fixed to the outside of the container or a part of the container other than the plunger. In such cases the non-retractable cutting element may be permanently fixed or may be removable.

As the first type of percutaneous drainage device shown in FIGS. 1C, 1D removes tissue, it may be referred to as a tissue retrieval device. While FIGS. 1C and 1D show the process for draining a shallow subcutaneous skin abscess, FIGS. 1F-1I show an example of a process for draining a deep skin abscess. In this case the skin abscess extends into the subcutaneous fat tissue; in some cases a deep skin abscess may be wholly contained in the subcutaneous fat tissue and may not be visible from the skin surface. As the deep subcutaneous skin abscess is deeper, e.g. extending 4 cm or more below the skin surface, it is more difficult to drain and two separate devices may be used.

A first step of the draining process is shown in FIG. 1F and uses the first type of device 100, as described above in relation to FIGS. 1C-1E and further examples of which are shown in FIGS. 2A-11, 12-15 and 16A to 18B. In this step the cutting edge and tissue retrieval component of the first device 102 are used to cut away one or more layers of tissue above the abscess. In the example of FIG. 1F, the epidermis layer and part of the dermis layer are cut away. However, some of the dermis layer may remain depending on the depth of the dermis layer (typically 4-5 mm) and the length of the cutting edge 208. In some examples the cutting edge may cut into the subcutaneous fat tissue, but in many cases this may be avoided for safety reasons, so as to minimise discomfort and the risk of cutting something other than fat tissue. The skin plug may then be removed as described above with reference to FIGS. 1C-1E. In some cases suction may be used to remove or extract tissue as well.

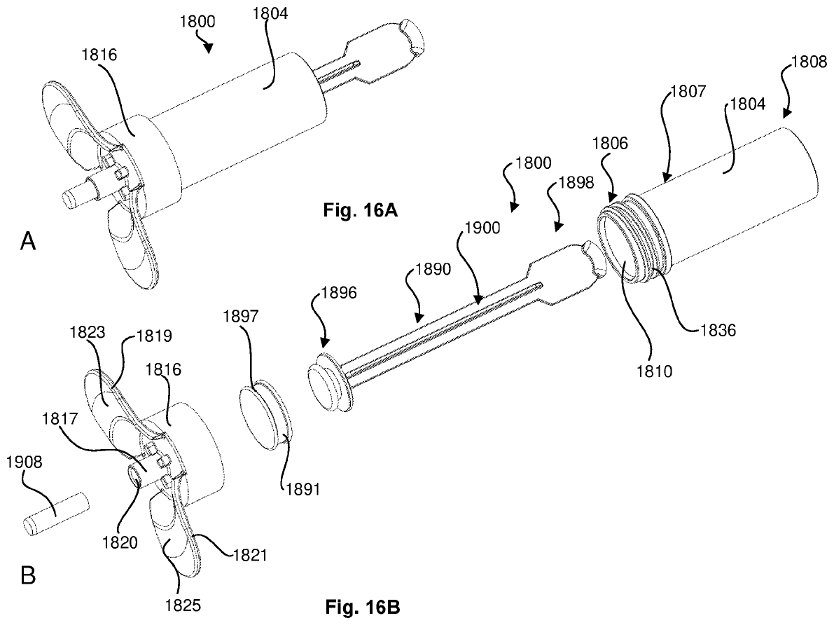

The subsequent steps shown in FIGS. 1G-1I use a second type of percutaneous drainage device 2200. The second type of percutaneous drainage device comprises a cannula 2202 which defines a lumen and a penetration component 2212 which is slidably engagable with the lumen of the cannula. The penetration component 2212 has a piercing end which can penetrate the subcutaneous fat tissue of the patient and be slid through the open end of the cannula to facilitate entry of the cannula into the patient. The cannula may have a thread on the outside to facilitate entry into the subcutaneous fat tissue by rotation. One example of the second type of percutaneous drainage device is shown in FIGS. 16-20.

In some examples, as shown in FIG. 1G, a guide wire 2228 may be introduced through the opening 12 formed by removal of the skin plug by the first device. The guide wire may be used to locate the abscess. Whether or not a guidewire is used, the second device 2200 may then be introduced to the opening and rotated or driven downwards into the tissue to make contact with the abscess. In cases where a guidewire is used, the penetration component may have an internal bore or lumen through which the guidewire can pass. A cap 2250 at an upper portion of the penetration component may be opened to allow passage of the guidewire. The guidewire may be removed through the bore and the cap 2250 may then be closed to seal of the bore. The penetration component 2212 may then be retracted and withdrawn through the cannula lumen. The penetration component may form a tight seal with the cannula lumen such that retraction of the penetration component through the cannula generates a negative pressure or vacuum which helps to start the flow of pus 3 or other material from the abscess into the cannula. As the cap 2250 is closed, the penetration component bore is sealed and the vacuum or negative pressure can be maintained as air cannot flow into the penetration component bore from above.

FIG. 1H shows the situation once the cannula 2202 has been driven down to the abscess and the penetration component 2212 has been withdrawn and removed. The pus or other contents of the abscess may now flow upwards out of the cannula and be collected in a bag or other collection vessel. Draining the abscess may take several hours or several days depending on the abscess size and contents. The cannula may have a collar 2254 which may be positioned on the skin surface to secure the cannula in place. If necessary, the cannula be cut to size by cutting off an end portion extending above the skin surface. As shown in FIG. 1I, a retention strap 2304 may be attached to the collar 2254 to secure the cannula 2202 in place. In this way the patient may go home and return later to have the retention strap, collar and cannula removed once the abscess has drained.

In some examples, the first device 100 and second device 2200 may be provided together as a system or kit of parts for use in draining a subcutaneous skin abscess. In some examples the first and second device may be provided together with a guide wire, collar and/or retention strap. These devices may be used, as described above, to drain shallow and/or deep subcutaneous skin abscesses in an out-patient setting, without the need for a hospital stay, in-patient surgery or general anaesthetic.

While general principles have been described above, several examples will be described in more detail below. It is to be understood that any of the examples below may incorporate one or more the features described above. Further, features of one example may be combined with or applied to another example, unless the disclosure explicitly states this is not the case or logic dictates otherwise.

### Part 2: Example of First Device 100

FIGS. 2A to 11B illustrate one example of a first type of device 100, according to some embodiments. The device 100 is configured to enable the removal of a portion of skin, epidermis and/or dermis tissue of a patient above a subcutaneous skin abscess and/or enable drainage of a subcutaneous skin abscess. In some embodiments, the device 100 is configured to enable the retrieval of a portion of tissue of a patient. Thus, the device 100 may be referred to as a percutaneous drainage device, tissue removal device or tissue retrieval device 100.

In some embodiments, the tissue retrieval device 100 is configured to be used to enable the retrieval and/or removal of a portion of the skin of a patient. The device 100 may be useful for, and will be described primarily in the context of draining a shallow skin abscess or improving access to a deeper skin abscess. Some abscesses can form deep within a patient's subcutaneous tissue. The tissue retrieval device 100 may be used to remove a portion of the skin, epidermal and dermal tissue layers of the patient over a subcutaneous abscess to improve the ease with which the abscess can be accessed, for example, for drainage.

It should, however, be understood that the device 100 may have utility in improving access to any subcutaneous area or any subcutaneous fluid collection and that the subcutaneous fluid collection may comprise liquid and/or gas. In the context of drainage of an abscess, ‘fluid collection’ refers to a collection of pus, liquefied tissue, solid tissue and/or any other associated material held within the confines of an tissue wall (e.g. a wall of a subcutaneous fluid collection).

The device 100 comprises a container 102. The container 102 comprises a container body 104. The container body 104 extends in a container longitudinal direction 105. The container body 104 comprises a first container body end portion 106. The first container body end portion 106 may be referred to as a lower container body end portion (or a lower end portion of the container body 104). The first container body end portion 106 may be referred to as a proximal container body end portion (or a proximal end portion of the container body 104). The container body 104 comprises a second container body end portion 108. The second container body end portion 108 may be referred to as an upper container body end portion (or an upper end portion of the container body 104). The second container body end portion 108 may be referred to as a distal container body end portion (or a distal end portion of the container body 104). The second container body end portion 108 may be referred to as the upper container body end portion because, in use, the second container body end portion 108 is higher than the first container body end portion 106 with respect to the patient's tissue. The second container body end portion 108 may be referred to as the distal container body end portion because, in use, the second container body end portion 108 is distal to the patient's tissue, compared to the first container body end portion 106 (i.e. the proximal container body end portion). The container body 104 comprises an intermediate container body portion 107. The intermediate container body portion 107 is between the first container body end portion 106 and the second container body end portion 108.

The container body 104 defines a first container body opening 110. The first container body opening 110 is disposed at the first container body end portion 106. The first container body opening 110 is a circular opening. It will be understood that in some embodiments, the first container body opening 110 may take another shape, such as a rounded rectangle. The first container body opening 110 may be referred to as a lower container body opening. The first container body opening 110 may be referred to as a proximal container body opening. The container body 104 defines a second container body opening 112. The second container body opening 112 is disposed at the second container body end portion 108. The second container body opening 112 is a circular opening. It will be understood that in some embodiments, the second container body opening 112 may take another shape, such as a rounded rectangle. The second container body opening 112 may be referred to as an upper container body opening (as, in use, the second container body opening 112 is higher than the first container body opening 110 with respect to the patient's tissue). The second container body opening 112 may be referred to as a distal container body opening as, in use, the second container body opening 112 is distal to the patient's tissue, compared to the first container body opening 110.

A container body lumen 114 extends between the first container body opening 110 and the second container body opening 112. In other words, the container body 104 defines the container body lumen 114. The container body lumen 114 extends from the first container body end portion 106 to the second container body end portion 108. The container body lumen 114 is generally cylindrical. That is, the container body lumen 114 is a cylindrical lumen. In some embodiments, the container body lumen 114 may have a different profile. For example, the container body lumen 114 may have a rectangular profile, a rounded rectangular profile, or a profile corresponding to another polygon. Alternatively, the container body lumen 114 may have an oval profile, or a profile corresponding to another curved shape. One or more features of the container body 104 may project into the container body lumen 114 at some areas. Thus, while generally cylindrical, some cross sectional profiles of the container body lumen 114 may include projections that result in the relevant cross sectional profiles deviating from a circular profile. The container body 104 comprises a container body inner surface 115. The container body inner surface 115 defines a cross sectional profile of the container body lumen 114 along its length. The container body inner surface 115 may be referred to as an inner surface of the container body 104. The container body inner surface 115 is an inner surface of the container 102.

The container body 104 may therefore be referred to as a hollow body. The container body 104 may be referred to as a barrel.

The tissue retrieval device 100 comprises a first cap 116. The first cap 116 may be referred to as a first container outlet cap. The first cap 116 is configured to connect to the container body 104. In particular, the first cap 116 is configured to removably connect to the container body 104. The first cap 116 is configured to connect to the first container body end portion 106.

The first cap 116 defines a first container outlet 118. In particular, the first cap 116 defines a first opening 120 and a second opening 122. A first cap lumen 124 extends between the first opening 120 and the second opening 122. The first container outlet 118 may correspond to some or all of the first cap lumen 124.

The tissue retrieval device 100 comprises a second cap 126. The second cap 126 may be referred to as a second container outlet cap. The second cap 126 is configured to connect to the container body 104. In particular, the second cap 126 is configured to removably connect to the container body 104. The second cap 126 is configured to connect to the second container body end portion 108.

The second cap 126 defines a second container outlet 128. In particular, the second cap 126 defines a first opening 130 and a second opening 132. A second cap lumen 134 extends between the first opening 130 and the second opening 132. The second container outlet 128 may correspond to some or all of the second cap lumen 134.

The container body 104 comprises a first container body connection portion 136. The first container body connection portion 136 is configured to enable the first cap 116 to connect to the container body 104. The first container body connection portion 136 comprises external threads of a threaded connection. The first cap 116 comprises internal threads of the threaded connection. It will be appreciated that in some embodiments, the first cap 116 may connect to the container body 104 using a different type of connection. For example, the first cap 116 may connect to the container body 104 using an interference fit connection, a snap fit connection or another type of connection.

The container body 104 comprises a second container body connection portion 138. The second container body connection portion 138 is configured to enable the second cap 126 to connect to the container body 104. The second container body connection portion 138 comprises external threads of a threaded connection. The second cap 126 comprises internal threads of the threaded connection. It will be appreciated that in some embodiments, the second cap 126 may connect to the container body 104 using a different type of connection. For example, the second cap 126 may connect to the container body 104 using an interference fit connection, a snap fit connection or another type of connection.

The container 102 comprises a container rim 140. In particular, the container body 104 comprises the container rim 140. The container rim 140 extends inwardly in a radial direction, towards a container longitudinal axis of the container 102. The illustrated container 102 comprises a first container rim 140A and a second container rim 140B. The first container rim 140A extends inwardly around a first portion of the inner perimeter of the container body 104. The second container rim 140B extends inwardly around a second portion of the inner perimeter of the container body 104.

In some embodiments, the container 102 comprises one container rim 140. The container rim 140 may extend inwardly in the radial direction of the container 102. The container rim 140 may extend around an entire inner perimeter of the container body 104.

The container 102 comprises an elongate projection 182. In particular, the container body 104 comprises the elongate projection 182. The elongate projection 182 extends inwardly in the radial direction of the container 102. That is, the elongate projection 182 extends towards a central longitudinal axis of the container body 104. The intermediate container body portion 107 comprises the elongate projection 182.

The elongate projection 182 comprises a first elongate portion 184. The elongate projection 182 comprises a second elongate portion 186. The second elongate portion 186 is a tapered portion of the elongate projection 182. That is, a thickness of the elongate projection 182 decreases along a length of the second elongate portion 186. The second elongate portion 186 is between the container rim 140 and the first elongate portion 184. In particular, the second elongate portion 186 is between the container rim 140 and the first elongate portion 184 in the container longitudinal direction 105.

As illustrated, in some embodiments, the container body 104 comprises a first elongate projection 182A and a second elongate projection 182B. The first elongate projection 182A may be on an opposing side of the container body 104 as the second elongate projection 182B.

The container 102 defines a containment volume 144. The container 102 defines a distal volume 146. The container rim 140 is between the containment volume 144 and the distal volume 146. The containment volume 114 is between the first container outlet 118 and the container rim 140. An inner container hole 142 fluidly connects the containment volume 144 and the distal volume 146. The first opening 120 of the first cap 116 and the second opening 132 of the second cap 126 are fluidly connected by the containment volume 144, the inner container hole 142 and the distal volume 146.

As described herein, in one or more embodiments, the first cap 116 and/or the second cap 126 are removably connectable with the container body 104. In some embodiments, the tissue retrieval device 100 comprises a first container cap (not shown). The tissue retrieval device 100 may also comprise a second container cap (not shown). The first container cap is configured to removably connect to the container body 104, as is described with reference to the first cap 116. However, the first container cap does not comprise a lumen. The first container cap is configured to connect to the container body 104 to obstruct the first container body opening 110, for example, to retain a fluid sample within the container 102, as is described herein. Similarly, the second container cap is configured to removably connect to the container body 104, as is described with reference to the second cap 126. However, the second container cap does not comprise a lumen. The second container cap is configured to connect to the container body 104 to obstruct the second container body opening 112, for example, to retain a fluid sample within the container 102, as is described herein.

The tissue retrieval device 100 may comprise a plunger sleeve 150. The plunger sleeve 150 comprises a first sleeve end portion 170. The first sleeve end portion 170 may be referred to as a lower sleeve end portion (or a lower end portion of the plunger sleeve 150). The first sleeve end portion 170 may be referred to as a proximal sleeve end portion (or a proximal end portion of the plunger sleeve 150). The plunger sleeve 150 comprises a second sleeve end portion 172. The second sleeve end portion 172 may be referred to as an upper sleeve end portion (or an upper end portion of the plunger sleeve 150). The second sleeve end portion 172 may be referred to as a distal sleeve end portion (or a distal end portion of the plunger sleeve 150).

The plunger sleeve 150 defines a first sleeve opening 152. The first sleeve opening 152 is disposed at the first sleeve end portion 170. The first sleeve opening 152 is a generally circular opening. It will be understood that in some embodiments, the first sleeve opening 152 may take another shape, such as a rounded rectangle. The first sleeve opening 152 may be referred to as a lower sleeve opening. The first sleeve opening 152 may be referred to as a proximal sleeve opening. The plunger sleeve 150 defines a second sleeve opening 154. The second sleeve opening 154 is disposed at the second sleeve end portion 172. The second sleeve opening 154 is a generally circular opening. It will be understood that in some embodiments, the second sleeve opening 154 may take another shape, such as a rounded rectangle. The second sleeve opening 154 may be referred to as an upper sleeve opening. The second sleeve opening 154 may be referred to as a distal sleeve opening.

A sleeve lumen 156 extends between the first sleeve opening 152 and the second sleeve opening 154. In other words, the plunger sleeve 150 defines the sleeve lumen 156. The sleeve lumen 156 extends from the first sleeve opening 152 to the second sleeve opening 154. The sleeve lumen 156 is generally cylindrical. In some embodiments, the sleeve lumen 156 may have a different profile. For example, the sleeve lumen 156 may have a rectangular profile, a rounded rectangular profile, or a profile corresponding to another polygon. Alternatively, the sleeve lumen 156 may have an oval profile, or a profile corresponding to another curved shape. The plunger sleeve 150 may therefore be referred to as a hollow body. The plunger sleeve 150 comprises a sleeve inner surface 157. The sleeve inner surface 157 defines a cross sectional profile of the sleeve lumen 156 along its length. The sleeve inner surface 157 may be referred to as an inner surface of the plunger sleeve 150.

The plunger sleeve 150 defines a plunger sleeve volume 151. The plunger sleeve volume 151 may correspond to the sleeve lumen 156.

The plunger sleeve 150 comprises an intermediate sleeve portion 160. The intermediate sleeve portion 160 is between the first sleeve end portion 170 and the second sleeve end portion 172. The sleeve lumen 156 comprises an intermediate sleeve lumen portion 168. The intermediate sleeve portion 160 defines the intermediate sleeve lumen portion 168. The intermediate sleeve lumen portion 168 is between the first sleeve opening 152 and the second sleeve opening 154.

The plunger sleeve 150 comprises a first sleeve connector member 158. The plunger sleeve 150 comprises an intermediate opening 160. In particular, the intermediate sleeve portion 160 comprises the intermediate opening 160. The intermediate opening 160 extends through a wall of the plunger sleeve 150 to fluidly connect the sleeve lumen 156 to a region exterior of the plunger sleeve 150. The first sleeve connector member 158 projects into the intermediate opening 160 from an intermediate opening wall 162. The first sleeve connector member 158 is an elongate member. The first sleeve connector member 158 is integrally formed with the plunger sleeve 150. The first sleeve connector member 158 is bendable.

The first sleeve connector member 158 comprises an elongate portion 159. The first sleeve connector member 158 comprises a first projection 164. The first projection 164 projects perpendicularly to a longitudinal direction of the first sleeve connector member 158. The first projection 164 projects inwardly in a radial direction of the plunger sleeve 150. The first projection 164 projects away from the elongate portion 159.

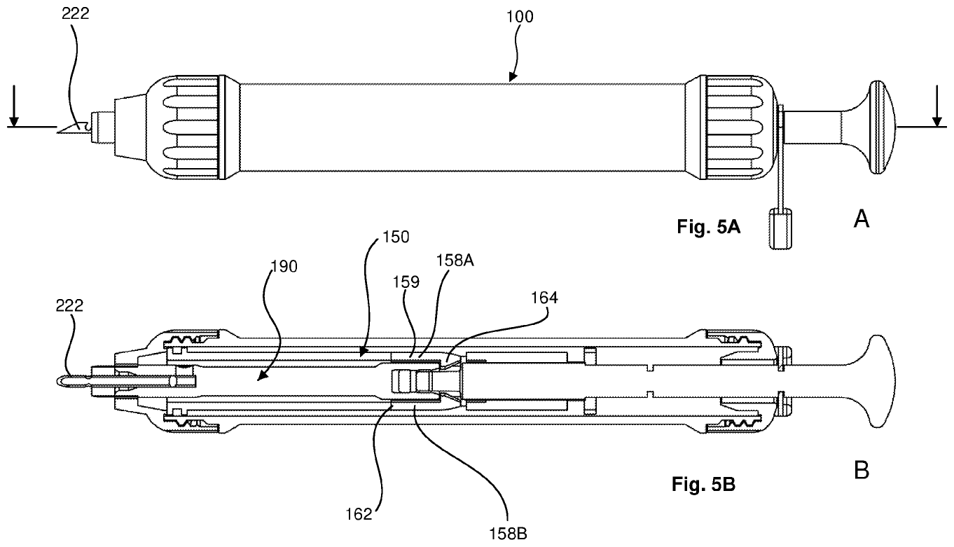

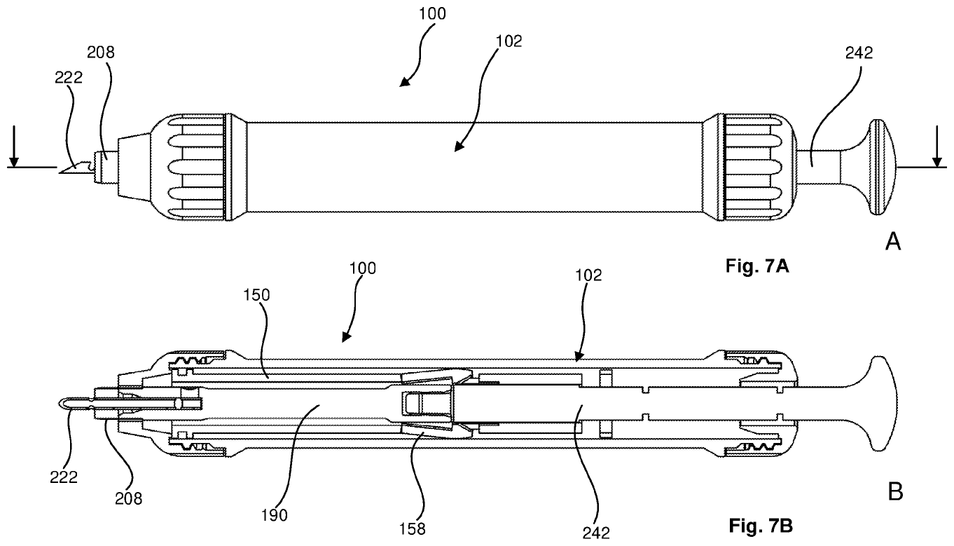

The first sleeve connector member 158 is moveable between a first position and a second position. The first sleeve connector member 158 is shown in the first position in FIG. 5A and FIG. 5B. The first sleeve connector member 158 is shown in the second position in FIG. 7A and FIG. 7B. The first sleeve connector member 158 is biased towards the first position. That is, in the absence of a significant force on the first sleeve connector member 158, the first sleeve connector member 158 moves towards, or remains in the first position.

As illustrated in FIGS. 2A to 11B, in some embodiments, the plunger sleeve 150 comprises a plurality of first sleeve connector members 158A, B. In some embodiments, the plunger sleeve 150 comprises a pair of first sleeve connector members 158A, B. Each of the first sleeve connector members 158A, B may be as described herein with respect to the first sleeve connector member 158. The first sleeve connector members 158A, B may be on opposing sides of the plunger sleeve 150. That is, the plunger sleeve 150 may comprise a pair of opposing first sleeve connector members 158A, B.

The plunger sleeve 150 comprises a second sleeve connector member 174. The plunger sleeve 150 comprises a second sleeve connector member opening 176. In particular, the second sleeve end portion 172 comprises the second sleeve connector member opening 176. The second sleeve connector member opening 176 may be a notch in the plunger sleeve 150. The second sleeve connector member 174 projects into the second sleeve connector member opening 176 from a second sleeve connector member opening wall 178. The second sleeve connector member 174 is an elongate member. The second sleeve connector member 174 is integrally formed with the plunger sleeve 150. The second sleeve connector member 174 is bendable.

The second sleeve connector member 174 comprises a second elongate portion 181. The second sleeve connector member 174 comprises a second projection 180. The second projection 180 projects perpendicularly to a longitudinal direction of the second sleeve connector member 174. The second projection 180 projects inwardly in a radial direction of the plunger sleeve 150. The second projection 180 projects away from the second elongate portion 181.

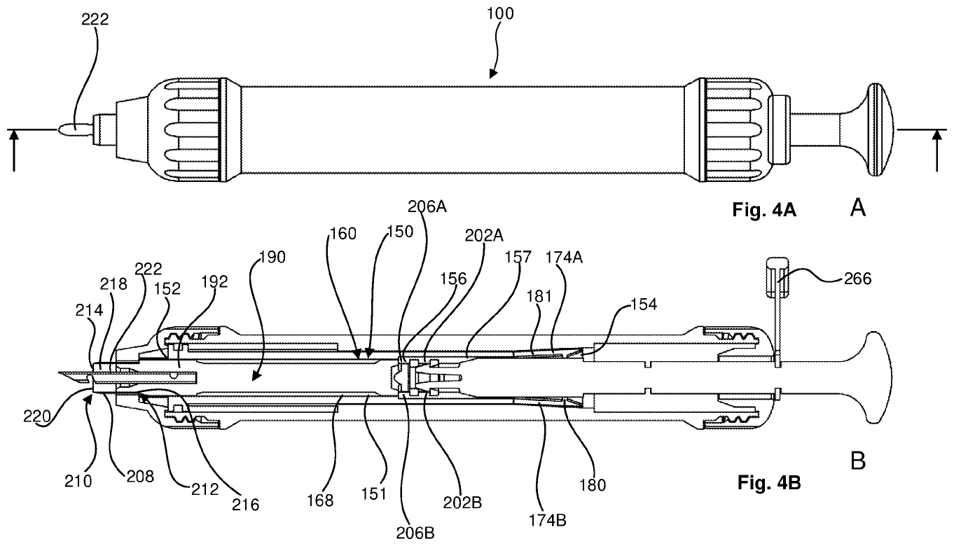

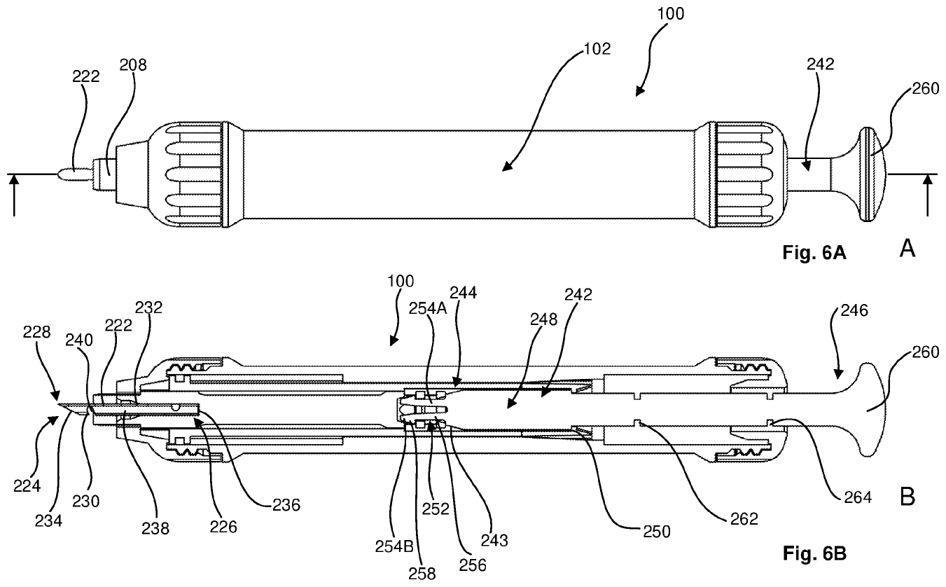

The second sleeve connector member 174 is moveable between a first position and a second position. The second sleeve connector member 174 is shown in the first position in FIG. 4A and FIG. 4B. The second sleeve connector member 174 is shown in the second position in FIG. 6A and FIG. 6B. The second sleeve connector member 174 is biased towards the second position. That is, in the absence of a significant force on the second sleeve connector member 174, the second sleeve connector member 174 moves towards, or remains in the second position.

As illustrated in FIGS. 2A to 11B, in some embodiments, the plunger sleeve 150 comprises a plurality of second sleeve connector members 174A, B. In some embodiments, the plunger sleeve 150 comprises a pair of second sleeve connector members 174A, B. Each of the second sleeve connector members 174A, B may be as described herein with respect to the second sleeve connector member 174. The second sleeve connector members 174A, B may be on opposing sides of the plunger sleeve 150. That is, the plunger sleeve 150 may comprise a pair of opposing second sleeve connector members 174A, B.

The plunger sleeve 150 comprises a groove 188. The groove 188 is an elongate grove that is parallel with a longitudinal direction of the plunger sleeve 150. The groove 118 is configured to receive at least part of the elongate projection 182 of the container body 104. The groove 188 and the elongate projection 182 together form a tongue and groove connection that enables relative movement between the plunger sleeve 150 and the container body 104 in the container longitudinal direction 105 while inhibiting relative movement of the plunger sleeve 150 and the container body 104 in one or more directions orthogonal to the container longitudinal direction 105.

The container 102 is configured to contain the plunger sleeve 150. In particular, the container 102 is configured to contain the plunger sleeve 150 within the containment volume 144.

The tissue retrieval device 100 comprises a plunger 190. The plunger sleeve 150 is configured to receive at least part of the plunger 190. In other words, the plunger sleeve 150 is configured to contain at least part of the plunger 190. The plunger 190 is configured to move in the container longitudinal direction 105 when received within the plunger sleeve 150. The container 102 is configured to contain at least part of the plunger 190. In other words, the plunger 190 is configured to be received by the container 102.

The plunger 190 comprises a first end portion 196. The first end portion 196 may be referred to as a lower end portion (or a lower end portion of the plunger 190). The first end portion 196 may be referred to as a proximal end portion (or a proximal end portion of the plunger 190). The plunger 190 comprises a second end portion 198. The second end portion 198 may be referred to as an upper end portion (or an upper end portion of the plunger 190). The second end portion 198 may be referred to as a distal end portion (or a distal end portion of the plunger 190). The plunger 190 comprises an intermediate plunger portion 200. The intermediate plunger portion 200 is between the first end portion 196 and the second end portion 198.

The plunger 190 comprises a sleeve contacting portion 192. In some embodiments, the sleeve contacting portion 192 is at the first end portion 196 of the plunger 190. In some embodiments, the sleeve contacting portion 192 is closer to the first end portion 196 of the plunger 190 than the second end portion 198 of the plunger 192. In some embodiments, the intermediate plunger portion 200 comprises the sleeve contacting portion 190. In some embodiments, the sleeve contacting portion is at the second end portion 198 of the plunger 190. In some embodiments, the sleeve contacting portion 192 is closer to the second end portion 198 of the plunger 190 than the first end portion 196 of the plunger 190.

The sleeve contacting portion 192 is configured to contact the plunger sleeve 150. In particular, the sleeve contacting portion 192 is configured to contact the sleeve inner surface 157. The sleeve contacting portion 192 is configured to slidably engage with the sleeve inner surface 157. That is, the plunger 190 is configured to be moved within the plunger sleeve 150 such that the sleeve contacting portion 192 slides along the sleeve inner surface 157. The plunger 190 is rotatable with respect to the plunger sleeve 150.

In some embodiments, the sleeve contacting portion 192 is configured to sealingly engage with the plunger sleeve 150. That is, the sleeve contacting portion 192 is configured to contact the sleeve inner surface 157 to impede movement of fluid through the interface between the sleeve contacting portion 192 and the sleeve inner surface 157.

In some embodiments the device may have a sealing component which is configured to seal with an interior chamber of the container and to generate suction when moved towards the proximal end when the tissue engagement component is retracted into the container. This can help draw tissue and/or viscous fluid out from the subcutaneous skin abscess and into the container. For example the sealing component may be an o-ring or a bulge on the plunger. The sealing component may seal with interior walls of the container or with a plunger sleeve where the device has a plunger sleeve.

In some embodiments, the sleeve contacting portion 192 of the sleeve comprises one or more of a polymeric material and elastomeric material. In some embodiments, the sleeve contacting portion 192 comprises an O ring. That is, the sleeve contacting portion 192 may comprise an O ring groove, and an O ring configured to be disposed in the O ring groove. The O ring may be configured to sealingly engage with the sleeve inner surface 157. In some embodiments, the O-ring comprises one or more of an elastomeric, polymeric, metallic and ceramic material. While O-rings are referred to as an example here, other types of sealing member could be used.

The plunger 190 comprises a first plunger connection portion 194. In particular, the second end portion 198 of the plunger 190 comprises the first plunger connection portion 194. The first plunger connection portion 194 defines a first plunger connection hole 202. In some embodiments, the first plunger connection portion 194 defines a plurality of first plunger connection holes 202. In the embodiment illustrated in FIGS. 1 to 11B, the first plunger connection portion 194 defines a pair of opposed first plunger connection holes 202A, B.

The plunger 190 comprises a second plunger connection portion 204. In particular, the second end portion 198 of the plunger 190 comprises the second plunger connection portion 204. The second plunger connection portion 204 defines a second plunger connection hole 206. In some embodiments, the second plunger connection portion 204 defines a plurality of second plunger connection holes 206. In the embodiment illustrated in FIGS. 1 to 11B, the second plunger connection portion 204 defines a pair of opposed second plunger connection holes 206A, B.

The tissue retrieval device 100 comprises a cutting element 208. The cutting element 208 is configured to cut tissue of the patient. In particular, the cutting element 208 is configured to cut the skin of the patient. The cutting element 208 may comprise a cylindrical cutting sharp. Thus, the tissue retrieval device 100 comprises the cylindrical cutting sharp. The cylindrical cutting sharp may be referred to as a biopsy punch. The cylindrical cutting sharp may be referred to as a biopsy sharp. The cutting element may have any of the dimensions or features described above in Part 1.

The cutting element 208 comprises a cutting end portion 210. The cutting element 208 comprises a second end portion 212. The second end portion 212 may be referred to as a second end portion of the cutting element 208. The second end portion 212 may be referred to as a distal end portion of the cutting element 208. The cutting element 208 comprises a cutting end opening 214. The cutting element 208 comprises a second end opening 216. The cutting element 208 extends from the cutting end portion 210 to the second end portion 212 to define a cutting lumen 218. The cutting lumen 218 extends between the cutting end opening 214 and the second end opening 216. The cutting lumen 218 extends in a longitudinal direction of the cutting element 208. The cutting element 208 comprises a cutting edge 220. The cutting edge 220 is sharpened to enable the cutting element 208 to cut tissue when the cutting element 208 is moved towards the tissue to contact the tissue. In particular, the cutting edge 220 enables the cutting element 208 to cut tissue when the cutting element 208 is moved towards and into the tissue in the longitudinal direction of the cutting element 208.

The cutting element 208 is configured to connect to the plunger 190. Similarly, the plunger 190 is configured to connect to the cutting element 208. In particular, the cutting element 208 is configured to connect to the first end portion 196 of the plunger 190. As illustrated in FIGS. 1 to 11B, the cutting element 208 is connected to the plunger 190. The longitudinal axis of the cutting element 208 may be coaxial with the longitudinal axis of the container 102. The longitudinal axis of the cutting element 208 may be coaxial with the longitudinal axis of the plunger 190. Movement of the plunger 190 causes movement of the cutting element 208.

In some embodiments, the cutting element 208 is configured to connect to the container 102. That is, rather than connecting to the plunger 190, the cutting element 208 may connect to the container 102. In particular, the cutting element 208 may connect to the first cap 116 at the first container outlet 118. The longitudinal axis of the cutting element 208 may be parallel with the container longitudinal direction 105.

The tissue retrieval device 100 comprises a tissue engagement component 222. The tissue engagement component 222 is configured to engage the tissue cut by the cutting element 208. In particular, the tissue engagement component 222 is configured to engage the tissue cut by the cutting element 208 such that movement of the tissue engagement component 222 causes movement of at least part of the tissue cut by the cutting element 208. The tissue engagement component 222 may have any of the dimensions or features described above in Part 1. The clearance between the tissue engagement component and the cutting element may be as described above in Part 1.

The tissue engagement component 222 comprises a penetrating end portion 224. The tissue engagement component 222 comprises a second end portion 226. The second end portion 226 may be referred to as a second end portion of the tissue engagement component 222. The second end portion 226 may be referred to as a distal end portion of the tissue engagement component 222. The tissue engagement component 222 is an elongate component that extends from the penetrating end portion 224 to the second end portion 226.

The tissue engagement component 222 comprises a penetration portion 228. In particular, the penetrating end portion 224 comprises the penetration portion 228. The penetration portion 228 is configured to penetrate tissue. In particular, the penetration portion 228 is configured to penetrate tissue prior to surrounding tissue being cut by the cutting element 208. The penetration portion 228 may be in the form of a sharpened portion of the tissue engagement component 222.

The tissue engagement component 222 comprises a barb 230. The barb 230 is configured to engage the tissue.

The tissue engagement component 222 comprises a shaft 232. The shaft 232 is an elongate portion of the tissue engagement component 222.

In some embodiments, the tissue engagement component 222 is solid. That is, the tissue engagement component 222 is not hollow.

In some embodiments, the tissue engagement component 222 defines a first tissue engagement component opening 234. The tissue engagement component 222 defines a second tissue engagement component opening 236. The tissue engagement component 222 extends from the penetrating end portion 224 to the second end portion 226 to define a tissue engagement component lumen 238. The tissue engagement component lumen 238 extends between the first tissue engagement component opening 234 and the second tissue engagement component opening 236. The tissue engagement component lumen 238 extends in a longitudinal direction of the tissue engagement component 222. The tissue engagement component 222 defines a barb opening 240. The barb opening 240 is fluidly connected to the tissue engagement component lumen 238.

The tissue engagement component 222 is configured to connect to the plunger 190. Similarly, the plunger 190 is configured to connect to the tissue engagement component 222. In particular, the tissue engagement component 222 is configured to connect to the first end portion 196 of the plunger 190. Thus, when connected, movement of the plunger 190 causes movement of the tissue engagement component 222. As illustrated in FIGS. 2A to 11B, the tissue engagement component 222 is connected to the plunger 190. The longitudinal axis of the tissue engagement component 222 may be parallel to the container longitudinal direction 105. The longitudinal axis of the tissue engagement component 222 may be coaxial with the longitudinal axis of the plunger 190. The longitudinal axis of the tissue engagement component 222 may be coaxial with the longitudinal axis of the cutting element 208.

The tissue engagement component 222 and the cutting element 208 are separate components. That is, the tissue engagement component 222 and the cutting element 208 are independently connectable to the plunger 190.

The tissue retrieval device 100 comprises a second plunger 242. In some embodiments, the plunger 190 may be referred to as a first plunger 190. The second plunger 242 is configured to connect to the plunger 190 such that, when connected, movement of the second plunger 242 causes corresponding movement of the plunger 190.

The second plunger 242 comprises a first end portion 244. The first end portion 244 may be referred to as a lower end portion (or a lower end portion of the second plunger 242). The first end portion 244 may be referred to as a proximal end portion (or a proximal end portion of the second plunger 242). The second plunger 242 comprises a second end portion 246. The second end portion 246 may be referred to as an upper end portion (or an upper end portion of the second plunger 242). The second end portion 246 may be referred to as a distal end portion (or a distal end portion of the second plunger 242). The second plunger 242 comprises an intermediate second plunger portion 248. The intermediate second plunger portion 248 may be referred to as an intermediate portion of the second plunger 242. The intermediate second plunger portion 248 is between the first end portion 244 and the second end portion 246. The second plunger 242 comprises a step 250. In particular, the second plunger 242 comprises the step 250 where the intermediate second plunger portion 248 meets the second end portion 246. An outer dimension of the intermediate second plunger portion 248 is larger than an outer dimension of the second end portion 246 to define the step 250. For example, in some embodiments, a perimeter of the intermediate second plunger portion 248 is larger than a perimeter of the second end portion 246 to define the step 250.

The second plunger 242 comprises an angled wall 243. A plane tangential to a surface of the angled wall 243 is transverse to a longitudinal direction of the second plunger 242.

The second plunger 242 comprises a second plunger connector 252. In particular, the first end portion 244 of the second plunger 242 comprises the second plunger connector 252. The second plunger connector 252 is configured to enable connection of the second plunger 242 and the plunger 190. In particular, the second plunger connector 252 is configured to enable the second plunger 242 to connect to the plunger 190.

The second plunger connector 252 comprises a second plunger connector member 254. The second plunger connector member 254 comprises an elongate portion 256. The elongate portion 256 may be referred to as an elongate portion of the second plunger connector member 254. The elongate portion 256 may be referred to as a second plunger connector member elongate portion. The second plunger connector member 254 comprises a projection 258. The projection 258 may be referred to as a projection of the second plunger connector member 254. The projection 258 may be referred to as a second plunger connector member projection. The projection 258 projects perpendicularly to a longitudinal direction of the second plunger connector member 254. The projection 258 projects outwardly in a radial direction of the second plunger 252.

As illustrated in FIGS. 2A to 11B, in some embodiments, the second plunger 242 comprises a plurality of second plunger connector members 254A, B. In some embodiments, the second plunger 242 comprises a pair of second plunger connector members 254A, B. Each of the second plunger connector members 254A, B may be as described herein with respect to the second plunger connector member 254. The second plunger connector members 254A, B may be on opposing sides of the second plunger 242. That is, the second plunger 242 may comprise a pair of opposing second plunger connector members 254A, B.

The second plunger 242 comprises an actuation portion 260. The actuation portion 260 is configured to enable a user to actuate the second plunger 242. Specifically, the actuation portion 260 is configured to enable the user to move the second plunger 242 with respect to the container 102.

The second plunger 242 comprises a first groove 262. In particular, the second end portion 246 to comprises the first groove 262. The first groove 262 extends circumferentially around the second plunger 242. The first groove 262 may act as an indicator to highlight that the plunger 242 has been moved to a retracted position. The second plunger 242 comprises a second groove 264. In particular, the second end portion 246 comprises the second groove 264. The second groove 264 extends circumferentially around the second plunger 242. The first groove 262 is between the actuation portion 260 and the intermediate second plunger portion 248. The second groove 264 is between the actuation portion 260 and the intermediate second plunger portion 248. The second groove 264 is between the actuation portion 260 and the first groove 262.

The tissue retrieving device 100 comprises a locking element 266. The locking element 266 is configured to engage the second groove 264. In particular, a portion of the locking element 266 is configured to be received within the second groove 264. When received within the second groove 264, the locking element 266 is configured to contact the container 102 to inhibit movement of the second plunger 242 with respect to the container 102. In particular, the locking element 266 is configured to inhibit movement of the actuation portion 260 towards the container 102. In some embodiments, the locking element 266 is configured to engage the first groove 262.

In use, the components of the tissue retrieval device 100 are movable between a plurality of positions. Movement of one or more of the components of the retrieval device 100 can result in the retrieval device 100 being moved between a plurality of states.

FIGS. 2A, 4A, 4B, 5A and 5B illustrate the tissue retrieval device 100 in a first state. The first state is a pre-use state. That is, the first state is the state in which the tissue retrieval device 100 is maintained prior to being used to retrieve tissue from a patient. In the first state, one or more of the components of the tissue retrieval device 100 are in a respective first state position. The first state may be referred to as an initial state. The first state may be referred to as an assembled state.

In the first state, the tissue engagement component 222 and the cutting element 208 are brought into contact with the patient's skin. The tissue retrieval device 100 is brought towards the patient's skin such that the tissue engagement component 222 penetrates the patient's skin, and the cutting element 208 cuts the patient's skin surrounding the tissue engagement component 222.

As described herein, the tissue retrieval device 100 comprises the container 102, the first plunger 190, the second plunger 242, the plunger sleeve 150, the cutting element 208 and the tissue engagement component 222. The container 102 is configured to contain at least part of the tissue engagement component 222. For example, as shown in FIGS. 2A, 4A, 4B, 5A and 5B, in the first state, the container 102 contains a part of the tissue engagement component 222. Specifically, the container 102 contains the second end portion 226 of the tissue engagement component 222. The container 102 also contains a part of the shaft 232.

As described herein, the tissue engagement component 222 is configured to engage the tissue cut by the cutting element 208. The tissue engagement component 222 is also configured to move with movement of the plunger 190. In some embodiments, the tissue engagement component 222 is configured to engage the tissue cut by the cutting element 208 such that movement of the tissue engagement component 222 into the containment volume 144 of the container 102 causes movement of at least part of the tissue cut by the cutting element into the containment volume 144.

The tissue engagement component 222 extends through the cutting lumen 218. In particular, the tissue engagement component 222 extends through the cutting lumen 218 such that the penetration portion 228 and the barb 230 are exposed beyond the cutting lumen 218. The tissue engagement component 222 extends through the first container outlet 118. The cutting element 208 extends through the first container outlet 118.

The plunger sleeve 150 is configured to contain at least part of the first plunger 190. The plunger sleeve 150 is configured to contain at least part of the second plunger 242. As shown in FIGS. 4A and 4B, in the first state, the plunger sleeve 150 is configured to receive the first plunger 190 in the sleeve lumen 156. Similarly, the plunger sleeve 150 is configured to receive the second plunger 242 in the sleeve lumen 156.

When the tissue retrieval device 100 is in the first state, the plunger 190 may be said to be in a first position. This may be a first position of the plunger 190. The first position may be referred to as an initial position of the plunger 190. The first position may be referred to as an assembled position of the plunger 190.

When the tissue retrieval device 100 is in the first state, the second plunger 242 may be said to be in a first position. This may be a first position of the second plunger 242. The first position may be referred to as an initial position of the second plunger 242. The first position may be referred to as an assembled position of the second plunger 242.

When the second plunger 242 is in the first position, the second plunger connector 252 is connected to the first plunger connection portion 194. In particular, the projection 258 of the second plunger connector member 254 is received within the first plunger connection hole 202. In other words, the first plunger connection hole 202 is configured to receive a portion of the second plunger connector member 254 when the second plunger 242 is in the first position. This inhibits relative movement of the second plunger 242 with respect to the plunger 190.

As shown in FIG. 5A and FIG. 5B, when the tissue retrieval device 100 is in the first state, the first sleeve connector member 158 is in the first position. In particular, each of the first sleeve connector members 158A, B are in their respective first position. In the first position, the first sleeve connector member 158 is configured to inhibit movement of the first plunger 190 with respect to the plunger sleeve 150. Specifically, the first sleeve connector member 158 is configured to abut an end of the first plunger 190 to inhibit movement of the first plunger 190 with respect to the plunger sleeve 150 when the first plunger 190 is moved away from the first container outlet 118. The first projection 166 of the first sleeve connector member 158 is configured to abut the end of the plunger 190.

As shown in FIGS. 4A and 4B, when the tissue retrieval device 100 is in the first state, the second sleeve connector member 174 is in the first position. In particular, each of the second sleeve connector members 174A, B are in their respective first position. In the first position, the second sleeve connector member 174 is configured to abut an outer surface of the second plunger 242.

The tissue retrieval device 100 is used to engage and cut the tissue of the patient when in the first state. The tissue retrieval device 100 is moved towards the tissue of the patient such that the container longitudinal direction 105 is generally perpendicular to the tissue of the patient. The container 102 is moved towards the tissue of the patient such that the penetration portion 228 penetrates the tissue of the patient. The tissue engagement component 222 engages the tissue at the barb 230. The locking element 266 may be engaged with the second groove 264 at this time. Further movement of the container 102 towards the tissue causes the cutting element 208 to cut the tissue surrounding the tissue engagement component 222.

FIGS. 6A to 7B illustrate the tissue retrieval device 100 in a second state. The second state is an intermediate state. That is, the second state is a state into which the tissue retrieval device 100 is moved, in use. The user moves the second plunger 242 towards the first container outlet 118 to move the tissue retrieval device 100 from the first state to the second state. In the second state, one or more of the components of the tissue retrieval device 100 is in a respective second state position. The second state may be referred to as an intermediate state. The second state may be referred to as a first intermediate state.

When the tissue retrieval device 100 is in the second state, the second plunger 242 may be said to be in a second position. This may be a second position of the second plunger 242. The second position may be referred to as an intermediate position of the second plunger 242. The second position may be referred to as a first intermediate position of the second plunger 242. The second position may be referred to as a plunger connection position. The second plunger 242 is configured to be moved between the first position and the second position.

When the second plunger 242 is in the second position, the second plunger connector 252 is connected to the second plunger connection portion 204 of the plunger 190. In particular, the projection 258 of the second plunger connector member 254 is received within the second plunger connection hole 206. In other words, the second plunger connection hole 206 is configured to receive a portion of the second plunger connector member 254 when the second plunger 242 is in the second position. The portion of the second plunger connector member 254 received by the second plunger connection hole 206 is the same portion of the second plunger connector member 254 that is received by the first plunger connection hole 202 when the second plunger 242 is in the first position. This inhibits relative movement of the second plunger 242 with respect to the plunger 190. In other words, movement of the second plunger 242 causes corresponding movement of the plunger 190.

As shown in FIGS. 7A and 7B, when the tissue retrieval device 100 is in the second state, the first sleeve connector member 158 is in the second position. In particular, each of the first sleeve connector members 158A, B are in their respective second position. In the second position, the first sleeve connector member 158 is configured to enable movement of the plunger 190 with respect to the plunger sleeve 150. Specifically, the first sleeve connector member 158 is configured to deflect outwards and abut an exterior surface of the second plunger 242 to enable movement of the plunger 190 with respect to the plunger sleeve 150 when the plunger 190 is moved away from the first container outlet 118. The first projection 166 of the first sleeve connector member 158 is configured to abut the second plunger 242, with movement of the second plunger 242 towards the first container outlet 118 to move the second plunger 242 from the first position to the second position, pushing the first sleeve connector member 158 radially outwards.

As shown in FIGS. 6A and 6B, when the tissue retrieval device 100 is in the second state, the second sleeve connector member 174 is in the second position. In particular, each of the second sleeve connector members 174A, B are in their respective second position. In the second position, the second sleeve connector member 174 is configured to abut the step 250. The second sleeve connector member 174 is configured to abut the step 250 to inhibit movement of the second plunger 242 with respect to the plunger sleeve 150 when the second plunger 242 is moved away from the first container outlet 118.

The user moves the second plunger 242 towards the first container outlet 118 to move the tissue retrieval device 100 from the first state to the second state after the tissue of the patient is sufficiently cut and engaged using the cutting element 208 and the tissue engagement component 222.

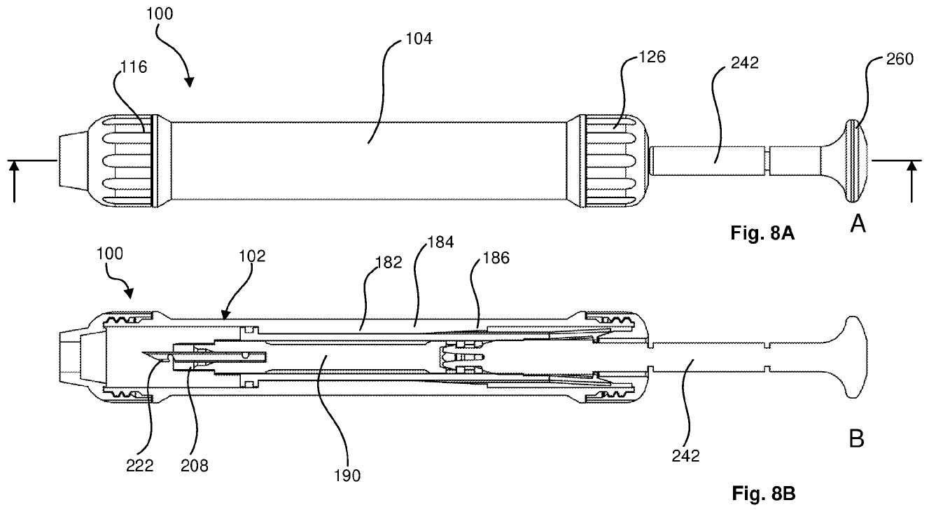

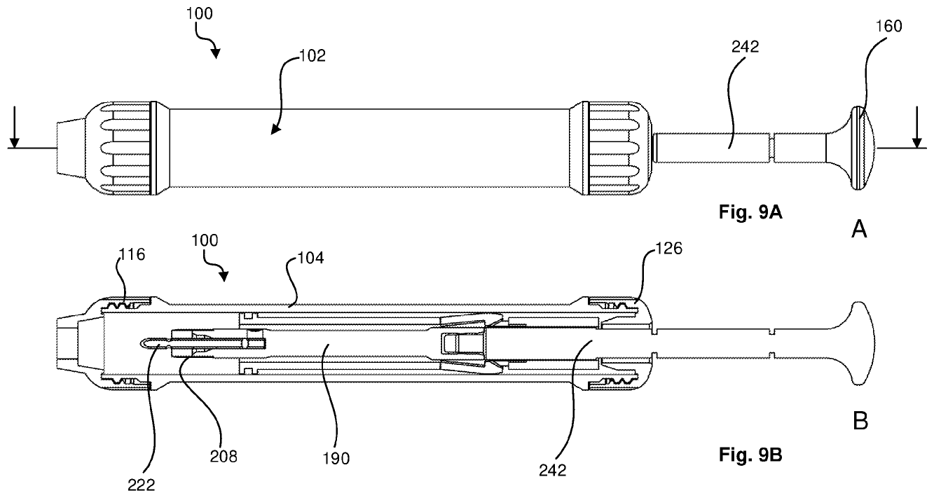

FIGS. 8A to 9B illustrate the tissue retrieval device 100 in a third state. The third state is an intermediate state. The third state may be referred to as a second intermediate state. The third state is a state into which the tissue retrieval device 100 is moved, in use. The user moves the second plunger 242 to move the tissue retrieval device 100 from the second state to the third state. In particular, the user moves the second plunger 242 away from the first container outlet 118 to move the tissue retrieval device 100 from the second state to the third state. In the third state, one or more of the components of the tissue retrieval device 100 is in a respective third state position. The user moves the second plunger 242 away from the first container outlet 118 to move the tissue retrieval device 100 from the second state to the third state.

The movement of the second plunger 242 away from the first container outlet 118 moves the tissue engagement component 222 and the cutting element 208 into the containment volume 144. This, in turn, moves the tissue cut by the cutting element 208 and engaged by the tissue engagement component 222 into the containment volume 144. As the described operations can be performed on tissue above an abscess, removal of the cut tissue may expose fluid of the abscess. The fluid may drain into the container 102. Furthermore, the movement of the second plunger 242 away from the first container outlet 118 may apply a negative pressure to the hole produced in the patient's tissue, thereby drawing the fluid into the container 102.

When the tissue retrieval device 100 is in the third state, the plunger 190 may be said to be in a second position. This may be a second position of the plunger 190. The second position may be referred to as an intermediate position of the plunger 190. The plunger 190 is configured to be moved between the first position and the second position.

When the plunger 190 is in the first position, at least part of the tissue engagement component 222 is exposed. That is, at least part of the tissue engagement component 222 extends outside the container 102. In particular, the first container outlet 118 is configured to receive the tissue engagement component 222 and the cutting element 208 such that at least part of the tissue engagement component 222 and at least part of the cutting element 208 extend outside of the container 102 through the first container outlet 118. When the plunger 190 is in the second position, the tissue engagement component 222 is contained within the containment volume 144 of the container 102. Furthermore, the cutting element 208 is contained within the containment volume 144.

When the tissue retrieval device 100 is in the third state, the second plunger 242 may be said to be in a third position. This may be a third position of the second plunger 242. The third position may be referred to as an intermediate position of the second plunger 242. The third position may be referred to as a second intermediate position of the second plunger 242. The third position may be referred to as a retracted position. The second plunger 242 is configured to be moved between the second position and the third position.

When the second plunger 242 is in the third position, the second plunger connector 252 is connected to the second plunger connection portion 204. Thus, the plunger 190 moves with movement of the second plunger 242.

As shown in FIGS. 9A and 9B, when the tissue retrieval device 100 is in the third state, the first sleeve connector member 158 is in the second position. In particular, each of the first sleeve connector members 158A, B are in their respective second position.

As the second plunger 242 is moved away from the first container outlet 118, the plunger sleeve 150 is also moved away from the first container outlet 118. This is because the step 250 of the second plunger 242 abuts the second projection 180 of the second sleeve connector member 174, causing the plunger sleeve 150 to move with movement of the second plunger 242. As the plunger sleeve 150 is moved away from the first container outlet 118, the second sleeve connector member 174 is moved past the second elongate portion 186.

When the tissue retrieval device 100 is in the third state, the second sleeve connector member 174 contacts the second cap 126. This inhibits further movement of the plunger sleeve 150 away from the first container outlet 118.

As shown in FIGS. 8A and 8B, when the tissue retrieval device 100 is in the third state, the second sleeve connector member 174 is in a third position. The third position generally corresponds to the first position. Thus, when the tissue retrieval device 100 is in the third state, the second sleeve connector member 174 reverts to the first position (i.e. is bent outwards). In particular, each of the second sleeve connector members 174A, B are in their respective first position. Moving the tissue retrieval device 100 from the second state to the third state causes the second sleeve connector member 174 to move from the second position to the third position. Specifically, moving the second plunger 242 away from the first container outlet 118 brings the second elongate portion 181 of the second sleeve connector member 174 into contact with an angled surface of the second cap 126. The angled surface is angled outwards from a centre of the second cap lumen 134. Thus, further movement of the second plunger 242 away from the first container outlet 118 causes the second sleeve connector member 174 to bend outwards, as a result of interaction with the angled surface of the second cap 126. This in turn releases the second plunger 242 from the plunger sleeve 150. In other words, longitudinal movement of the intermediate second plunger portion 248 away from the first container outlet 118 causes the second sleeve connector member 174 to move from the second position to the first position.

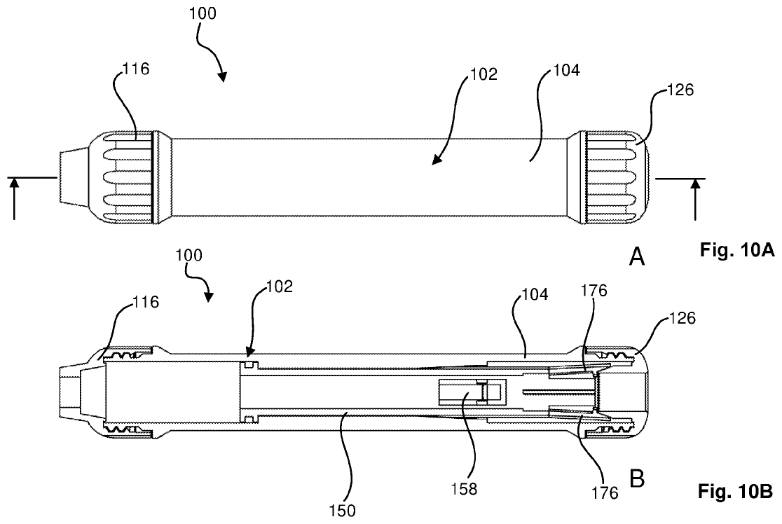

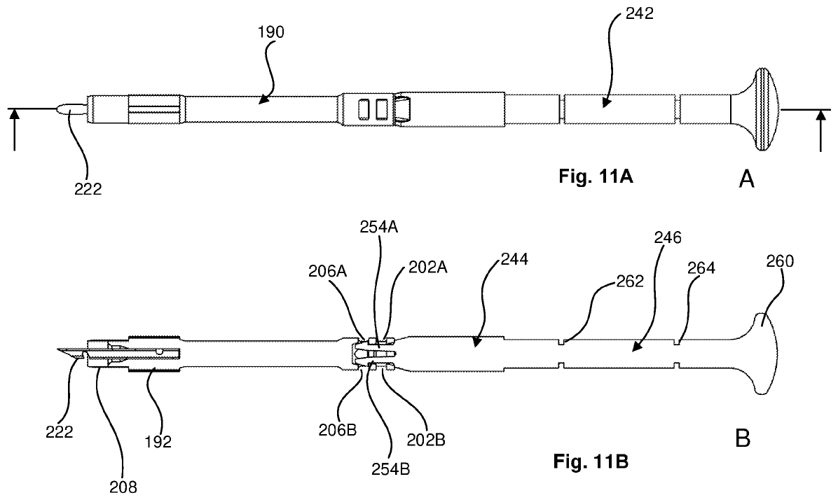

FIGS. 10A to 11B illustrate the tissue retrieval device 100 in a fourth state. The fourth state is an end state. The fourth state is a state into which the tissue retrieval device 100 is moved, in use. The user moves the second plunger 242 away from the first container outlet 118 to move the tissue retrieval device 100 from the third state to the fourth state. In the fourth state, one or more of the components of the tissue retrieval device 100 are in a respective fourth state position. Movement of the second plunger 242 away from the first container outlet 118 draws the second plunger 242 and the plunger 190 out of the second opening 132 of the second cap 126. This separates the second plunger 242 and the plunger 190 from the container 102. The plunger sleeve 150 abuts the second cap 126, and therefore remains within the container 102.

When the tissue retrieval device 100 is in the fourth state, the plunger 190 may be said to be in a third position. This may be a third position of the plunger 190. The third position may be referred to as an external position of the plunger 190.

When the tissue retrieval device 100 is in the fourth state, the second plunger 242 may be said to be in a fourth position. This may be a fourth position of the second plunger 242. The fourth position may be referred to as an external position of the second plunger 242.

Following separation of the plunger 190 and the second plunger 242 from the container 102, the first cap 116 and the second cap 126 may be removed. The first container cap may be connected to the first container body end portion 106, to obstruct the first container body opening 110. The second container cap may be connected to the second container body end portion 108 to obstruct the second container body opening 112, and retain the fluid within the container 102.

In some embodiments, rather than being removed and replaced with another set of caps, the first cap 116 and the second cap 126 are plugged. That is, a first plug may be connected to the first cap 116 to obstruct the first container outlet 118. Similarly, a second plug may be connected to the second cap 126 to obstruct the second container outlet 128.

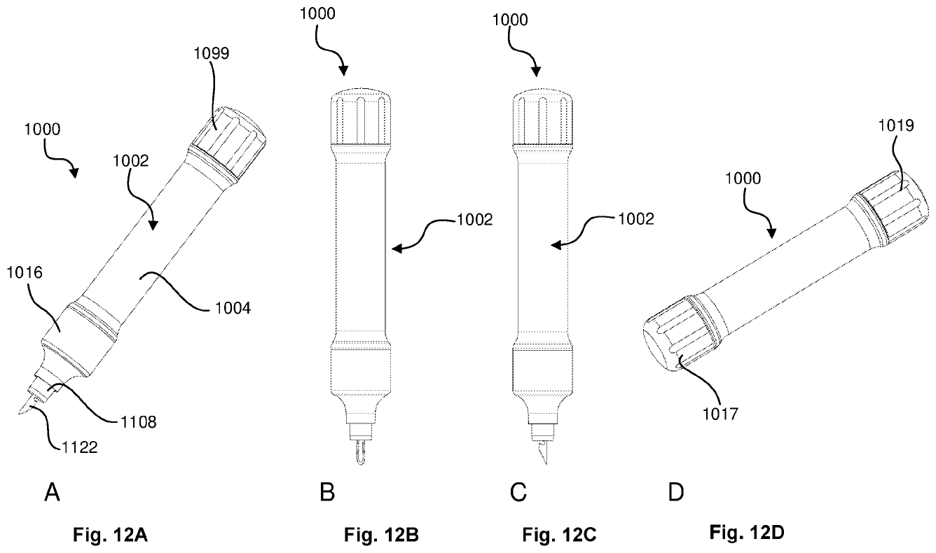

FIGS. 12A to 15B illustrate another embodiment of the tissue retrieval device 1000, according to some embodiments.

Whereas, in the embodiment of FIGS. 2A to 11B, the cutting element is mounted to the plunger, in the embodiment of FIGS. 12A to 15B, the cutting element 1108 is mounted to the container 1002. Specifically, the cutting element 1108 is mounted to the first cap 1016 of the container 1002. The cutting element 1108 extends away from the container body 1104, such that part of the cutting element 1108 is within the first container outlet 1018 and part of the cutting element 1108 is exposed outside the first container outlet 1018.

A result of this difference is that the cutting element 1108 of the embodiment of the tissue retrieval device 1000 of FIGS. 12A to 15B, the cutting element 1108 is not drawn into the containment volume 1044 when the plunger is pulled away from the first container outlet 1018 when moving the tissue retrieval device 1000 from the second state to the third state. The tissue retrieval device 1000 of FIGS. 12A to 15B may be otherwise be similar to or same as the tissue retrieval of FIGS. 2A to 11B.

### Part 3: Further Example of First Device 1800

FIGS. 16A to 18B illustrate a tissue retrieval device 1800, according to some embodiments. The tissue retrieval device 1800 is configured to enable the retrieval of a portion of tissue of a patient. The tissue retrieval device 1800 is also configured to enable the retrieval of subcutaneous fluid of the patient. In some embodiments, the tissue retrieval device 1800 is configured to enable the removal of a portion of tissue of a patient. Thus, the tissue retrieval device 1800 may be referred to as a tissue removal device 1800.

In some embodiments, the tissue retrieval device 1800 is configured to be used to enable the retrieval and/or removal of a portion of the skin of a patient. The tissue retrieval device 1800 may be useful for, and will be described primarily in the context of improving access to an abscess. Some abscesses can form relatively deep within a patient's skin, for example, in the patient's subcutaneous tissue. Targeted access to such an abscess can be difficult using surgical tools such as scalpels. The tissue retrieval device 1800 is configured to enable the removal of a portion of the skin of the patient over a subcutaneous abscess to improve the ease with which the abscess can be accessed, for example, for drainage.

It should, however, be understood that the tissue retrieval device 1800 may have utility in improving access to any subcutaneous area m any subcutaneous fluid collection and that the subcutaneous fluid collection may comprise liquid and/or gas. In the context of drainage of an abscess, ‘fluid collection’ refers to a collection of pus, liquefied tissue and any other associated liquid held within the confines of an abscess wall.

The tissue retrieval device 1800 comprises a container 1802. The container 1802 may be similar to, or the same as one or more of the containers described herein, in one or more aspects. The container 1802 comprises a container body 1804. The container body 1804 extends in a container longitudinal direction 1805. The container body 1804 comprises a first container body end portion 1806. The container body 1804 comprises a second container body end portion 1808. The container body 1804 comprises an intermediate container body portion 1807. The intermediate container body portion 1807 is between the first container body end portion 1806 and the second container body end portion 1808.

The container body 1804 defines a first container body opening 1810. The first container body opening 1810 is disposed at the first container body end portion 1806. The container body 1804 defines a second container body opening 1812. The second container body opening 1812 is disposed at the second container body end portion 1808.

A container body lumen 1814 extends between the first container body opening 1810 and the second container body opening 1812. In other words, the container body 1804 defines the container body lumen 1814. The container body lumen 1814 extends from the first container body end portion 1806 to the second container body end portion 1808. The container body lumen 1814 is generally cylindrical. That is, the container body lumen 1814 is a cylindrical lumen. In some embodiments, the container body lumen 1814 may have a different profile. For example, the container body lumen 1814 may have a rectangular profile, a rounded rectangular profile, or a profile corresponding to another polygon. Alternatively, the container body lumen 1814 may have an oval profile, or a profile corresponding to another curved shape. One or more features of the container body 1804 may project into the container body lumen 1814 at some areas. Thus, while generally cylindrical, some cross sectional profiles of the container body lumen 1814 may include projections that result in the relevant cross sectional profiles deviating from a circular profile. The container body 1804 comprises a container body inner surface 1815. The container body inner surface 1815 defines a cross sectional profile of the container body lumen 1814 along its length.

The tissue retrieval device 1800 comprises a cap 1816. The cap 1816 is configured to connect to the container body 1804. The cap 1816 is configured to removably connect to the first container body end portion 1806. The cap 1816 comprises a cap collar 1817. The cap collar 1817 defines a first container outlet 1818. In particular, the cap 1816 defines a first opening 1820 and a second opening 1822. A first cap lumen 1824 extends between the first opening 1820 and the second opening 1822. The first container outlet 1818 may correspond to the first cap lumen 1824. The cap collar 1817 extends outwards, away from the second opening 1822.

The cap 1816 comprises one or more stabilising members. In the illustrated embodiment, the cap 1816 comprises a pair of opposed stabilising members 1819, 1821. In particular, the cap 1816 comprises a first stabilising member 1819 and a second stabilising member 1821. The first stabilising member 1819 extends away from the second opening 1822 and radially outward with respect to a longitudinal axis of the container 1802. The first stabilising member 1819 may be said to be an elongate stabilising member. An outer surface 1823 of the first stabilising member 1819 is configured to contact the patient's skin to stabilise the tissue retrieval device 1800 during use. The second stabilising member 1821 extends away from the second opening 1822 and radially outward with respect to a longitudinal axis of the container 1802. The second stabilising member 1821 may be said to be an elongate stabilising member. An outer surface 1825 of the second stabilising member 1821 is configured to contact the patient's skin to stabilise the tissue retrieval device 1800 during use.

The container body 1804 comprises a container body connection portion 1836. The container body connection portion 1836 is configured to enable the cap 1816 to connect to the container body 1804. The cap 1816 comprises a cap connecting portion 1837. The cap connecting portion 1837 is configured to enable the cap 1816 to connect to the container body 1804. The container body connection portion 1836 comprises external threads of a threaded connection. The cap connecting portion 1837 comprises internal threads of the threaded connection. It will be appreciated that in some embodiments, the cap connecting portion 1837 may connect to the container body 1804 using a different type of connection. For example, the cap connecting portion 1837 may connect to the container body 1804 using an interference fit connection, a snap fit connection or another type of connection.

The container 1802 comprises a container rim 1840. In particular, the container body 1804 comprises the container rim 1840. The container rim 1840 extends inwardly around an inner perimeter of the container body 1804. The container rim 1840 may extend around an entire inner perimeter of the container body 1804.

The container 1802 defines a containment volume 1844. The container 1802 defines a distal volume 1846. The container rim 1840 is between the containment volume 1844 and the distal volume 1846. The containment volume 1844 is between the first container outlet 1810 and the container rim 1840. An inner container hole 1842 fluidly connects the containment volume 1844 and the distal volume 1846, through the container rim 1840. The first opening 1820 of the cap 1816 and the second container 1812 are fluidly connected by the containment volume 1844, the inner container hole 1842 and the distal volume 1846.

The tissue retrieval device 1800 comprises a cutting element 1908. The cutting element 1908 is configured to cut tissue of the patient. The cutting element 1908 may be the same as or similar to any one of the cutting elements described herein. Thus, the cutting element may comprise a cylindrical cutting sharp.

The cutting element 1908 comprises a cutting end portion 1910. The cutting element 1908 comprises a second end portion 1812. The cutting element 1908 comprises a cutting end opening 1914. The cutting end opening 1914 may be referred to as a cutting element opening. The cutting element 1908 comprises a second end opening 1916. The cutting element 1908 extends from the cutting end portion 1910 to the second end portion 1912 to define a cutting lumen 1918. The cutting lumen 1918 extends between the cutting end opening 1914 and the second end opening 1916. The cutting element 1908 comprises a cutting edge 1920. The cutting edge 1920 is sharpened to enable the cutting element 1908 to cut tissue when the cutting element 1908 is moved towards the tissue to contact the tissue.

The cutting element 1908 is configured to connect to the container 1802. In particular, the cutting element 1908 is configured to connect to the cap 1816. The cutting element 1908 is configured to be mounted to the cap collar 1817. The cutting element 1908 connects to the cap 1817 at the first opening 1820.

The tissue retrieval device 1800 comprises a plunger 1890. The container 1802 is configured to receive at least part of the plunger 1890. In other words, the container 1802 is configured to contain at least part of the plunger 1890. The plunger 1890 is configured to move in the container longitudinal direction 1805.

The plunger 1890 comprises a first end portion 1896. A dimension of the first end portion 1896 is greater than a dimension of the inner container hole 1842. As a result, the first end portion 1896 of the plunger 1890 will not pass through the inner container hole 1842. The plunger 1890 comprises a second end portion 1898. The second end portion 1898 comprises an actuation portion 1899. The plunger 1890 comprises an intermediate plunger portion 1900. The intermediate plunger portion 1900 is between the first end portion 1896 and the second end portion 1898.

The plunger 1890 comprises a plunger sealing surface 1891. In some embodiments, the plunger 1890 is configured to sealingly engage with the container 1802. That is, the plunger 1890 is configured to contact the container body inner surface 1815 to impede movement of fluid through the interface between the portion of the plunger 1890 that contacts the container body inner surface 1822 and the container body inner surface 1822. In other words, the plunger sealing surface 1891 is configured to seal with the container body inner surface 1822 to inhibit fluid flow between the plunger sealing surface 1891 and the container body inner surface 1822.

In some embodiments, the relevant portion of the plunger 1890 comprises one or more of a polymeric material and elastomeric material 1897. In some embodiments, the relevant portion of the plunger 1890 comprises an O ring. That is, the plunger 1890 may comprise an O ring groove, and an O ring configured to be disposed in the O ring groove. The O ring may be configured to sealingly engage with the container body inner surface 1815. Thus, the O-ring may comprise the plunger sealing surface 1891. In some embodiments, the O-ring comprises one or more of an elastomeric, polymeric, metallic and ceramic material. The plunger 1890 is configured to be moved within the containment volume 1844. In particular, the plunger 1890 is configured to be moved in the container longitudinal direction 1805, within the containment volume 1844.

In use, the components of the tissue retrieval device 1800 are movable between a plurality of positions. Movement of one or more of the components of the retrieval device 1800 can result in the retrieval device 1800 being moved between a plurality of states.

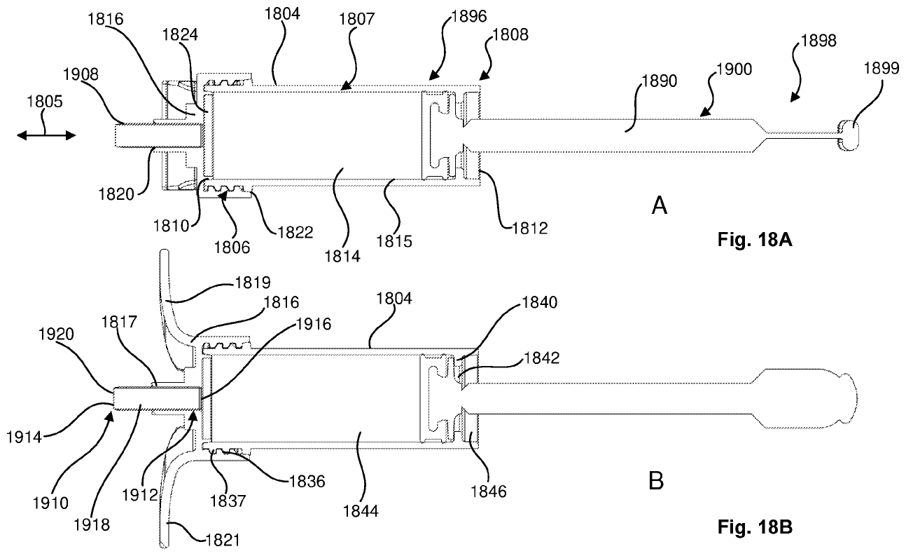

FIG. 18A illustrates a cross-sectional side view of the tissue retrieval device 1800 in a first state. In the first state, the plunger 1890 may be said to be in a first position. In the first position, the plunger 1890 is closer to the second container body end portion 1808 than the first container body end portion 1806.

The user moves the plunger 1890 to the first container body end portion 1806. This may be referred to as a second state of the tissue retrieval device 1800.

The user then moves the tissue retrieval device 1800 towards the patient's skin where tissue is to be retrieved. The user brings the cutting edge 1920 of the cutting element 1908 into contact with the patients skin and simultaneously pushes and rotates tissue retrieval device 1800 so that the cutting element 1908 cuts the patient's skin. The user pushes the tissue retrieval device 1800 towards the patient's skin until the cap collar 1817 abuts the patient's skin. The stabilising members 1819, 1821 may also contact the patient's skin to stabilise the tissue retrieval device 1800.

In some examples, the stabilisers may be between 10 and 25 mm so as to not dig in to the patient but also not be cumbersome. The stabilisers may have an oval shape main body which contacts the patient. The stabilisers may be formed from a material which is firm, but flexible enough to adapt to the skin and not be uncomfortable for the patient, such as but not limited to polypropylene. The diameter of the container bore may, for example, be between 10 and 15 mm. In some examples the container may have a length of 4 cm to 8 cm so as to contain the removed tissue and drained pus and material from the abscess.

As the plunger sealing surface 1891 is sealingly engaged with the container body inner surface 1815, a seal is formed between the patient's skin, the end of the cap collar 1817 and the plunger 1890. The suction pressure is a negative pressure. The suction pressure draws the tissue cut by the cutting element 1908 (and also, in some embodiments, fluid from the relevant abscess) into the containment volume 1844.

The user may cap the container 1802 as described herein.

The device 1800 may be modified to have a tissue engagement component similar to that described in Parts 1 and 2. The device of Part 2 may be modified to have stabilisers as described above for the device 1800.

### Part 4: Example of Second Device 2200

FIG. 19 illustrates an exploded perspective view of a second device, which may be used as a percutaneous drainage device 2200, according to some embodiments. Specifically, as explained above with reference to FIGS. 1F-1I, the second device may be used to drain a deeper subcutaneous skin abscess, after a first device has been used to cut away a skin plug formed from the epidermal layer and at least part, but not necessarily all of, the dermal layer above the subcutaneous abscess.

Thus, the percutaneous drainage device 2200 has application in drainage of a subcutaneous fluid collection from a patient. The percutaneous drainage device 2200 is useful for and will be described primarily in the context of draining an abscess. However, it should be understood that the percutaneous drainage device 2200 of the present disclosure has utility in gaining access to and drainage of any subcutaneous fluid collection and that the subcutaneous fluid collection may be liquid and/or gas.

The percutaneous drainage device 2200 comprises a cannula 2202. The cannula defines a cannula lumen through which pus and other fluid or contents or the subcutaneous skin abscess may be drained. The cannula lumen should have a diameter sufficiently large that viscous fluid does not easily block the cannula. In some examples the cannula lumen may have a diameter of 3 mm to 4 mm. The cannula 2202 comprises a first cannula end portion 2203. The first cannula end portion 2203 may be referred to as a lower cannula end portion (or a lower end portion of the cannula 2202). The first cannula end portion 2203 may be referred to as a proximal cannula end portion (or a proximal end portion of the cannula 2202). The cannula 2202 comprises a second cannula end portion 2205. The second cannula end portion 2205 may be referred to as an upper cannula end portion (or an upper end portion of the cannula 2202). The second cannula end portion 2205 may be referred to as a distal cannula end portion (or a distal end portion of the cannula 2202). The second cannula end portion 2205 may be referred to as the upper cannula end portion because, in use, the second cannula end portion 2205 is higher than the first cannula end portion 2203 with respect to the subcutaneous fluid collection. The second cannula end portion 2205 may be referred to as the distal cannula end portion because, in use, the second cannula end portion 2205 is distal to the subcutaneous fluid collection, compared to the first cannula end portion 2203. The cannula 2202 comprises an intermediate cannula portion 2207. The intermediate cannula portion 2207 is between the first cannula end portion 2203 and the second cannula end portion 2205.

The cannula 2202 defines a first cannula opening 2204. The first cannula opening 2204 may be referred to as a proximal cannula opening as, in use, the first cannula opening 2204 is proximal to the subcutaneous fluid collection. The cannula 2202 defines a second cannula opening 2206. The second cannula opening 2206 may be referred to as a distal cannula opening as, in use, the second cannula opening 2206 is distal to the subcutaneous fluid collection, when compared to the first cannula opening 2204.

A cannula lumen 2208 extends between the first cannula opening 2204 and the second cannula opening 2206. In other words, the cannula 2202 defines the cannula lumen 2208. The cannula lumen 2208 extends from the first cannula end portion 2203 to the second cannula end portion 2205. The cannula lumen 2208 is generally cylindrical. In some embodiments, the cannula lumen 2208 may have a different profile. For example, the cannula lumen 2208 may have a rectangular profile, a rounded rectangular profile, or a profile corresponding to another polygon. Alternatively, the cannula lumen 2208 may have an oval profile, or a profile corresponding to another curved shape. The cannula 2202 comprises a cannula inner surface 2210. The cannula inner surface 2210 defines a cross sectional profile of the cannula lumen 2208 along its length. The cannula inner surface 2210 may be referred to as an inner surface of the cannula 2202.

The cannula 2202 comprises a cannula flange 2240. The cannula flange 2240 defines the second cannula opening 2206. The second cannula end portion 2205 comprises the cannula flange 2240. The cannula 2202 comprises a recess 2242. In particular, the cannula flange 2240 comprises the recess 2242. The recess 2242 may be referred to as a cannula recess. In the illustrated embodiment, the cannula flange 2240 comprises a plurality of recesses 2242.

The cannula 2202 defines a cannula thread 2246. That is, at least part of an outer surface 2248 of the cannula 2202 may be threaded, providing the cannula thread 2246. The cannula thread 2246 may be referred to as a threaded surface. In the embodiment shown in the Figures, a majority of the outer surface 2248 of the cannula 2202 is threaded. The cannula thread 2246 can assist drilling and driving of the percutaneous drainage device 2200 into patient tissue.

The percutaneous drainage device 2200 comprises a penetration component 2212. The penetration component 2212 comprises a first penetration component end portion 2214. The first penetration component end portion 2214 may be referred to as a lower penetration component end portion (or a lower end portion of the penetration component 2212). The first penetration component end portion 2214 may be referred to as a proximal penetration component end portion (or a proximal end portion of the penetration component 2212). The penetration component 2212 comprises a second penetration component end portion 2216. The second penetration component end portion 2216 may be referred to as an upper penetration component end portion (or an upper end portion of the penetration component 2212). The second penetration component end portion 2216 may be referred to as a distal penetration component end portion (or a distal end portion of the penetration component 2212). The penetration component 2212 comprises an intermediate penetration component portion 2218. The intermediate cannula portion 2218 is between the first penetration component end portion 2214 and the second penetration component end portion 2216.

The penetration component 2212 defines a first penetration component opening 2220. The first penetration component opening 2220 may be referred to as a proximal penetration component opening. The penetration component 2212 defines a second penetration component opening 2222. The second penetration component opening 2222 may be referred to as a distal penetration component opening.

A penetration component lumen 2224 extends between the first penetration component opening 2220 and the second penetration component opening 2222. In other words, the penetration component 2212 defines the penetration component lumen 2224. The penetration component lumen 2224 extends from the first penetration component end portion 2214 to the second penetration component end portion 2216. The penetration component lumen 2224 is generally cylindrical. In some embodiments, the penetration component lumen 2224 may have a different profile. For example, the penetration component lumen 2224 may have a rectangular profile, a rounded rectangular profile, or a profile corresponding to another polygon. Alternatively, the penetration component lumen 2224 may have an oval profile, or a profile corresponding to another curved shape. The penetration component 2212 comprises a penetration component inner surface 2226. The penetration component inner surface 2226 defines a cross sectional profile of the penetration component lumen 2224 along its length. The penetration component inner surface 2226 may be referred to as an inner surface of the penetration component 2212. The penetration component lumen 2224 is configured to receive a guide wire 2228.

The penetration component 2212 comprises a penetration portion 2230. The penetration portion 2230 is configured to penetrate tissue of the patient, thereby enabling introduction of the first cannula opening 2204 to the subcutaneous fluid collection site. The penetration portion 2230 comprises a thread 2232. The thread 2232 is tapered towards a penetrating end 2234 of the penetration component 2212. The thread 2232 enables the user to drill and drive into the body of the patient when the penetration component 2212 is rotated about its longitudinal axis, as is described herein.

The penetration component 2212 comprises a shaft 2236. The shaft 2236 is an elongate shaft.

The penetration component 2232 comprises a gripping portion 2238. The gripping portion 2238 is configured to be gripped by a user. The gripping portion 2238 may be referred to as a handle. The gripping portion 2238 defines the second penetration component opening 2222. The shaft 2236 is between the penetration portion 2230 and the gripping portion 2238.

The penetration component lumen 2224 comprises a first portion 2225 and a second portion 2227. The penetrating portion 2230 and the shaft 2236 define the first portion 2225. The gripping portion 2238 defines the second portion 2227. A cross-sectional area of the second portion 2227 is greater than a cross-sectional area of the first portion 2225. In other words, a cross-sectional area of the penetration component lumen 2224 defined by the gripping portion 2238 is greater than a cross-sectional area of the penetration component lumen 2224 defined by the penetrating portion 2230 and the shaft 2236. As a result, the second penetration component opening 2222 is larger than the first penetration component opening 2220.

In some embodiments, the penetration portion 2230 may be referred to as a piercing portion. This may be because, in some embodiments, the penetration portion 2230 is configured to pierce the patient's skin.

The penetration component 2212 comprises a projecting feature 2244. The projecting feature 2244 projects from the gripping portion 2238 of the penetration component 2212, towards the penetration portion 2230.

The penetration component 2212 is configured to be received by the cannula 2202. The recess 2242 of the cannula 2202 is configured to receive the projecting feature 2244 when the penetration component 2212 is received within the cannula 2202, thereby impeding rotation of the cannula 2202 with respect to the penetration component 2212. The penetration component 2212 comprises a projecting feature 2244 for each recess of the cannula 2202.

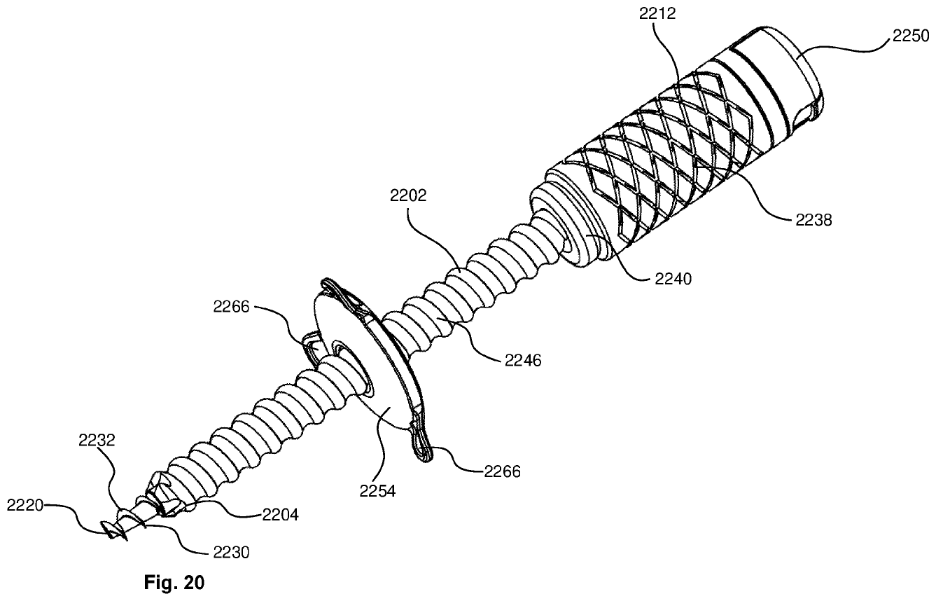

The penetration component 2212 is configured to be slidably received within the cannula 2202. The penetration component 2212 is moveable between a first position and a second position. When in the first position, the penetration component 2212 is received by the cannula 2202, which is concentric to the shaft 2236 of the penetration component 2212. In the first position, the penetration component 2212 extends through the first cannula opening 2204 and the second cannula opening 2206 (e.g. as shown in FIG. 20). When in the second position, the penetration component 2212 is retracted from and separated from the cannula 2202. In the first position, the penetration component 2212 may be located substantially inside the cannula lumen 2208. The penetration component 2212 may be prevented from rotating inside the cannula lumen 2208 by the interaction between the recesses 2242 and the projecting features 2244. The penetration component 2212 may have a tight tolerance with the cannula lumen 2208.

The percutaneous drainage device 2200 comprises a penetration component cap 2250. The penetration component cap 2250 is configured to removably engage with the penetration component 2212. In particular, the penetration component cap 2250 is configured to removably engage with the second penetration component end portion 2216. The penetration component cap 2250 enables selective obstruction of the second penetration component opening 2222.

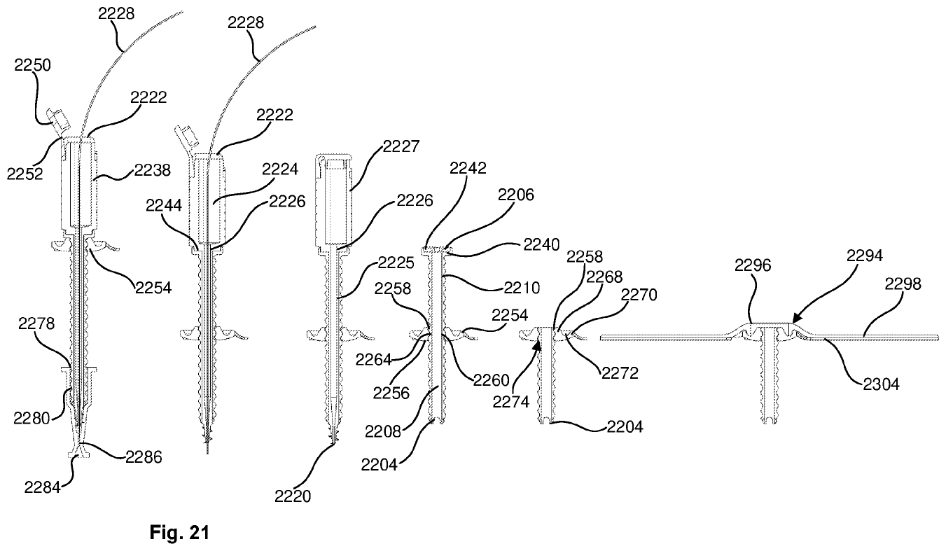

As illustrated in FIG. 21, the penetration component cap 2250 is moveable between a first position in which the second penetration component opening 2222 is obstructed and a second position where the second penetration component opening 2222 is unobstructed. The penetration component cap 2250 may be rotated about a flexible joint 2252 to be moved between the first position and the second position. The penetration component cap 2250 may connect to the penetration component 2212 when in the first position via an interference fit, snap fit or another type of connection.

The percutaneous drainage device 2200 comprises a collar 2254. The collar 2254 is configured to removably engage with the cannula 2202. The collar 2254 is configured to be moveable in a longitudinal direction of the cannula 2202, when engaged with the cannula 2202. The collar 2254 defines a first collar opening 2256. The first collar opening 2256 may be referred to as a proximal collar opening. The collar 2254 defines a second collar opening 2258. The second collar opening 2258 may be referred to as a distal collar opening.

A collar lumen 2260 extends between the first collar opening 2256 and the second collar opening 2258. In other words, the collar 2254 defines the collar lumen 2260. The collar lumen 2260 extends from the first collar opening 2256 to the second collar opening 2258. The collar lumen 2260 is generally cylindrical. In some embodiments, the collar lumen 2260 may have a different profile. For example, the collar lumen 2260 may have a rectangular profile, a rounded rectangular profile, or a profile corresponding to another polygon. Alternatively, the collar lumen 2260 may have an oval profile, or a profile corresponding to another curved shape. The collar 2254 comprises a collar inner surface 2262. The collar inner surface 2262 defines a cross sectional profile of the collar lumen 2260 along its length. The collar inner surface 2262 may be referred to as an inner surface of the collar 2254.

As described herein, the cannula 2202 defines a cannula thread 2246. The cannula thread 2246 is an external thread. The collar 2254 defines a collar thread 2264. The collar thread 2264 is an internal thread. That is, at least part of the collar inner surface 2262 may be threaded, providing the collar thread 2264. The collar thread 2264 may be referred to as a threaded surface. In the embodiment shown in the Figures, a majority of the collar inner surface 2262 is threaded.

The collar thread 2264 is configured to cooperate with the cannula thread 2246 to enable relative movement between the collar 2254 and the cannula 2202. When the collar lumen 2260 is aligned with the cannula 2202 (i.e. a longitudinal axis of the cannula lumen 2208 is coaxial with a longitudinal axis of the collar lumen 2260) and the collar 2254 is brought into contact with the first cannula end portion 2203, rotation of the collar 2254 with respect to the cannula 2202 can cause the collar to engage the cannula 2202. Specifically, the collar thread 2264 engages the cannula thread 2246 to enable longitudinal movement of the collar 2254 with respect to the cannula 2202. That is, rotation of the collar 2254 with respect to the cannula 2202 causes movement of the collar 2254 in the longitudinal direction of the cannula 2202. Movement of the collar 2254 in the longitudinal direction of the cannula 2202 is impeded without rotation of the collar 2254 with respect to the cannula 2202, as the collar thread 2254 contacts the cannula thread 2246.

The collar 2254 defines a peripheral hole 2266. In particular, the collar 2254 defines a plurality of peripheral holes 2266. In some embodiments, a user may use the peripheral holes 2266 to suture the collar to the skin of the patient.

The collar 2254 comprises an inner channel wall 2268. The inner channel wall 2268 is configured to contact the cannula 2202 when the collar 2254 is engaged with the cannula 2202. The collar 2254 comprises an outer channel wall 2270. The collar 2254 defines a channel 2272. In particular, the collar 2254 defines the channel 2272 between the inner channel wall 2268 and the outer channel wall 2270. The channel 2272 is an annular channel. The channel 2272 extends around at least a portion of the collar lumen 2260. The inner channel wall 2268 extends further away from the first collar opening 2256 than the outer channel wall 2270. In other words, a height of the inner channel wall 2268 is greater than a height of the outer channel wall 2270.

The percutaneous drainage device 2200 comprises a cap 2274. The cap 2274 defines a first cap opening 2276. The first cap opening 2276 may be referred to as a proximal cap opening. The cap 2274 defines a second cap opening 2278. The second cap opening 2278 may be referred to as a distal cap opening.

A cap lumen 2280 extends between the first cap opening 2276 and the second cap opening 2278. In other words, the cap 2274 defines the cap lumen 2280. The cap lumen 2280 extends from the first cap opening 2276 to the second cap opening 2278. The cap lumen 2280 is generally cylindrical. In some embodiments, the cap lumen 2280 may have a different profile. For example, the cap lumen 2280 may have a rectangular profile, a rounded rectangular profile, or a profile corresponding to another polygon. Alternatively, the cap lumen 2280 may have an oval profile, or a profile corresponding to another curved shape. The cap 2274 comprises a cap inner surface 2282. The cap inner surface 2282 defines a cross sectional profile of the cap lumen 2280 along its length. The cap inner surface 2282 may be referred to as an inner surface of the cap 2274. The cap lumen 2280 is configured to receive the cannula 2202 and the penetration component 2212, as described herein.

The cap lumen 2280 comprises a first tapered portion 2284. The first tapered portion 2284 extends from the first cap opening 2276 towards the second cap opening 2278. A cross-sectional area of the first tapered portion 2284 decreases as the first tapered portion 2284 extends from the first cap opening 2276 towards the second cap opening 2278.

The cap lumen 2280 comprises a guide portion 2286. The guide portion 2286 extends from the first tapered portion 2284 towards the second cap opening 2278. A cross sectional area of the guide portion 2286 may be constant.

The cap lumen 2280 comprises a second tapered portion 2288. The second tapered portion 2288 extends from the guide portion 2286 towards the second cap opening 2278. A cross-sectional area of the second tapered portion 2288 increases as the second tapered portion 2288 extends from the guide portion 2286 towards the second cap opening 2278 (i.e. away from the first cap opening 2276). The guide portion 2286 is between the first tapered portion 2284 and the second tapered portion 2288.

The cap lumen 2280 comprises a third tapered portion 2290. The third tapered portion 2290 extends from the second tapered portion 2288 towards the second cap opening 2278. A cross-sectional area of the third tapered portion 2290 increases as the second tapered portion 2288 extends from the second tapered portion 2288 towards the second cap opening 2278 (i.e. away from the first cap opening 2276).

The cap 2274 comprises a cap groove 2292. The cap groove 2292 extends from the first cap opening 2276 to the second cap opening 2278. The cap groove 2292 is configured to enable the passage of the guide wire 2228 from the cap lumen 2280 to outside of the cap 2274. In other words, a thickness of the cap groove 2292 is greater than a thickness of the guide wire 2228. The cap groove 2292 may be referred to as a longitudinal groove.

The percutaneous drainage device 2200 comprises a connecting component 2294. The connecting component 2294 comprises a collar engaging portion 2296. The collar engaging portion 2296 is configured to removably engage with the collar 2254. The collar engaging portion 2296 defines a collar engaging portion opening 2300. The collar engaging portion opening 2300 is configured to enable passage of the penetration component 2212. The collar engaging portion opening 2300 is configured to enable passage of the cannula 2202. In other words, a dimension of the collar engaging portion opening 2300 (e.g. a diameter) is greater than a corresponding dimension (e.g. a diameter) of the penetration component 2212 and the cannula 2202. The collar engaging portion 2296 comprises a collar engaging projection 2302. The collar engaging projection 2302 is configured to be received by the collar 2254 to inhibit relative movement between the collar 2254 and the collar engaging portion 2296. The collar engaging projection 2302 is an annular projection. The collar engaging projection 2302 is configured to be received by the channel 2272 of the collar 2254.

In some embodiments, the collar engaging portion 2296 comprises a locating projection (not shown). The locating projection may be configured to be received by one of the peripheral holes 2266 of the collar 2254 to inhibit rotation of the collar engaging portion 2296 with respect to the collar 2254.

The connecting component 2294 comprises an extension portion 2298. The extension portion 2298 extends radially from the collar engaging portion 2296. The extension portion 2298 is configured to connect to the tissue (e.g. the skin) of the patient. The extension portion 2298 comprises an adhesive portion 2304. The adhesive portion 2304 is configured to adhere to the patient. In particular, the adhesive portion 2304 is configured to adhere to the skin of the patient.

FIG. 21 illustrates a sequence of steps of use of the percutaneous drainage device 2200. It will be understood that the sequence of steps may vary depending on clinician preferences.

The subcutaneous fluid collection site (e.g. an abscess) or other fluid collection present in the patient is first located by the clinician. Following administration of a local anaesthetic around the fluid collection site, the guide wire 2228 is directed towards the fluid collection site. The guide wire 228 may be positioned using the Seldinger technique.

The clinician may assemble the percutaneous drainage device 2200. That is, the clinician may put the percutaneous drainage device 2200 in an assembled state. In the assembled state, the collar 2254 is engaged with the cannula 2202. Specifically, the collar 2254 is engaged with the cannula thread 2246 such that the inner channel wall 2268 contacts the cannula thread 2246 and the cannula flange 2240. In other words, the collar 2254 is screwed up the length of the cannula 2202 to the cannula flange 2240. The penetration component 2212 is received by the cannula 2202. Specifically, the penetration component 2212 is received by the cannula 2202 through the second cannula opening 2206, such that the penetration portion 2230 extends out of the first cannula opening 2204 and beyond the cannula 2202. The penetration component cap 2250 may be opened, thereby opening the second penetration component opening 2222. A portion of the assembled cannula 2202 and penetration component 2212 is received within the cap 2274. The first tapered portion 2284 of the cap 2274 is exposed, ready to receive an end of the guide wire 2228, and to guide the guide wire 2228 through the penetration component lumen 2224. In this state, the first penetration component opening 2220 is aligned with the guide portion 2286 of the cap lumen 2280. In other words, the first penetration component opening 2220 is aligned with the guide portion 2286 of the cap lumen 2280 when the penetration component 2212 is received in the cap 2274.

The clinician may guide the guide wire 2228 through the cap lumen 2280 from the first cap opening 2276, and through the penetration component lumen 2224 from the first penetration component opening 2220 to the second penetration component opening 2222.

The clinician may remove the cap 2274. Specifically, the clinician may remove the cannula 2202 and penetration component 2212 from the cap 2274, and then remove the cap 2274 from the guide wire 2228 by passing the guide wire 2228 through the cap groove 2292.

The clinician may then commence driving the percutaneous drainage device 2200 into the tissue. Specifically, the clinician may place the penetration portion 2230 in contact with the patient's tissue, apply a force in a longitudinal direction of the penetration component 2212, towards the tissue, and rotate the gripping portion 2238. The cannula 2202 will rotate with rotation of the gripping portion 2238, due to the cooperation of the recess 2242 and the projecting feature 2244.

The user may be assisted in driving and positioning of the percutaneous drainage device 2200 into the patient by imaging guidance, such as ultrasound or fluoroscopy. The thread 2232 of the penetration component 2212 assists the driving function of the percutaneous drainage device 2200, converting torsional energy into linear motion. Advantageously, this can provide greater positional control whilst simultaneously reducing need for downwards force into the tissue. As the penetration component 2212 is driven into the tissue, the thread 2232 gently stretches cutaneous tissue onto the cannula 2202 which may assist in reducing site morbidity. Threading on the surface of the cannula 2202 may also assist the percutaneous drainage device 2200 to drive further into the tissue and towards the target fluid collection site.

The clinician may determine that the first cannula opening 2204 has reached the target fluid collection site by imaging guidance. Once the first cannula opening 2204 has reached the abscess or target fluid collection site, the clinician may grasp the gripping portion 2238 of the penetration component 2212 and pull away from the body of the patient to retract the penetration component 2212, removing it from the cannula 2202 through the second cannula opening 2206. The clinician may further confirm that the first cannula opening 2204 appropriately positioned in the target fluid collection site by visually observing presence of pus or other fluid upon retraction of the penetration component 2212. The tight tolerance between the penetration component 2212 and cannula 2202 can generate a negative pressure during retraction of the penetration component 2212 from the cannula 2202, initiating suction and flow of fluid collection from the abscess and into the cannula lumen 2208. The cannula 2202 provides an extraction pathway through which the fluid contents of the target fluid collection site may drain from the patient's body.

The collar 2254 can now be moved longitudinally with respect to the cannula 2202 by rotating the collar 2254 with respect to the cannula 2202 in the appropriate direction. The collar 2254 can be moved towards the patient's skin, to contact the patient's skin, thereby assisting in holding the cannula 2202 in the appropriate position.

The length of the cannula 2202 can be trimmed as required. The cannula 2202 may be cut to size using a bladed instrument. The cannula 2202 may be trimmed so that an upper end of the cannula is flush with an upper face of the collar 2254. Alternatively, a relatively short length of the cannula 2202 may extend above the upper face of the collar 2254. The cannula 2202 is flexible.

The clinician may then connect the connecting component 2294. The clinician may align the collar engaging projection 2302 with the channel 2272 of the collar 2254 and bring the connecting component 2294 into contact with the collar 2254 such that the collar engaging projection 2302 is received by the channel 2272. The clinician may adhere the connecting component 2294 to the patient's skin using the adhesive portions 2304. In this way, the cannula 2202 is anchored to the patient's skin.

Once it has been determined that the fluid collection has been sufficiently drained, the cannula 2202 may be removed from the patient. All components of the percutaneous drainage device 2200 may be discarded following use.

The percutaneous drainage device 2200 of the present disclosure, when placed percutaneously substantially as described above, advantageously provides a channel and passage for thick, complex collections comprising any combination of pus, debris and dead tissue, to flow out from within the subcutaneous abscess.

The percutaneous drainage device 2200 of the present disclosure conveniently provides all the components required to offer a minimally invasive therapy that can be used to treat an abscess or another fluid collection. The cannula 2202 can be positioned with high precision using the guide wire 2228 and a clinician using the percutaneous drainage device 2200 can quickly and easily drain an abscess or other similar subcutaneous fluid collection in a setting which does not require hospital admission. The percutaneous drainage device 2200 of the present disclosure therefore advantageously shifts the treatment of abscesses and other subcutaneous fluid collections from a high-cost setting requiring hospital inpatient admission, theatre time and associated patient risk, to a relatively low-cost outpatient procedure with reduced clinical care time and associate patient risk.

### Alternative Percutaneous Drainage Device Embodiments

Although the percutaneous drainage device 2200 is described herein as being used with the guide wire 2228, it will be appreciated that guide wires are not universally available, and the percutaneous drainage device 2200 may be used without a guide wire. Therefore, in some embodiments, the penetration component 2212 does not comprise the penetration component lumen 2224.

### Fluid Collection Drainage System

A fluid collection drainage system may be provided by the present disclosure. In particular, the fluid collection drainage system may comprise any one of the described tissue retrieval devices 100, 300, 600, 800, 1000, 1200, 1400, 1600, 1800 and/or 2000, and the percutaneous drainage device 2200 described herein.

There are a number of advantages of using one of the described tissue retrieval devices with the described percutaneous drainage device 2200. As described herein, the tissue retrieval devices enable the removal of tissue (e.g. skin) above a fluid collection. Some fluid collections can be deep within the patient. The described tissue retrieval devices enable the removal of tougher layers of the patient's skin above an abscess. The percutaneous drainage device 2200 then enables a user to direct the described cannula 2202 through deeper layers of skin to the cavity of the abscess. The described tissue retrieval devices enable the provision of a hole that is the smallest optimal size for the cannula 2202. That is, the hole is large enough that it can receive the cannula 2202, but small enough that there is minimal excess size after receipt of the cannula 2202, to assist in minimising disfigurement and scaring of the patient. Furthermore, the size of the hole is optimised to prevent the hole from healing too fast (i.e. before the abscess is drained).

In some cases, it may be difficult to accurately position the cannula 2202 within the fluid collection, or it may be difficult to drive the percutaneous drainage device 2200 through the skin of the patient to a large enough depth accurately enough. In at least these cases, one of the described tissue retrieval devices can be used to retrieve tissue above the fluid collection, prior to using the percutaneous drainage device 2200. This can reduce the amount of tissue through which it is necessary to drive the percutaneous drainage device 2200, increasing the ease with which the fluid collection may be accessed, and decreasing the likelihood that the percutaneous drainage device 2200 will be driven into the patient in an incorrect or unoptimized orientation.

A first device as described in Parts 2 or 3 may be provided together with a second device as described in Part 4 as a set or system for treating subcutaneous skin abscesses. In some cases the first and second devices may be provided together with a guidewire.

Two possible variations of the first device will now be described. While the first device in Parts 1 and 2 has been described as having a tissue engagement component in a harpoon like shape comprising a shaft, penetration portion and barb, in a first variation, the tissue engagement component may instead of a shaft, penetration portion and barb comprise a pair of jaws or tongues which are to grab a skin plug, after the skin plug has been cut out by the cutting element. The jaws may have a sharp pointed distal end to penetrate the tissue and may have one or more teeth for grabbing the tissue.

In a second variation, instead of a shaft, penetration portion and barb, the tissue engagement component may instead take the form of a cylindrical component with one or more shark teeth. The cylindrical component may define a lumen and may comprise one or more apertures and a tapered projection (or shark tooth) adjacent each aperture, wherein the tapered projection projects radially inward into the lumen and points back towards a proximal end of the tissue engagement component away from a distal end of the cutting element. The tapered projection(s) may thus act to engage with and hold onto a skin plug which has been cut by the cutting element, so when the tissue engagement component is retracted into the container, the skin plug cut by the cutting element is pulled back into the container. The shark tooth may act as barbs to engage with and pull the skin plug back into the container. In other respects, the first device according to these variations may similar or the same as the device described above in Parts 1 and 2 of the description.

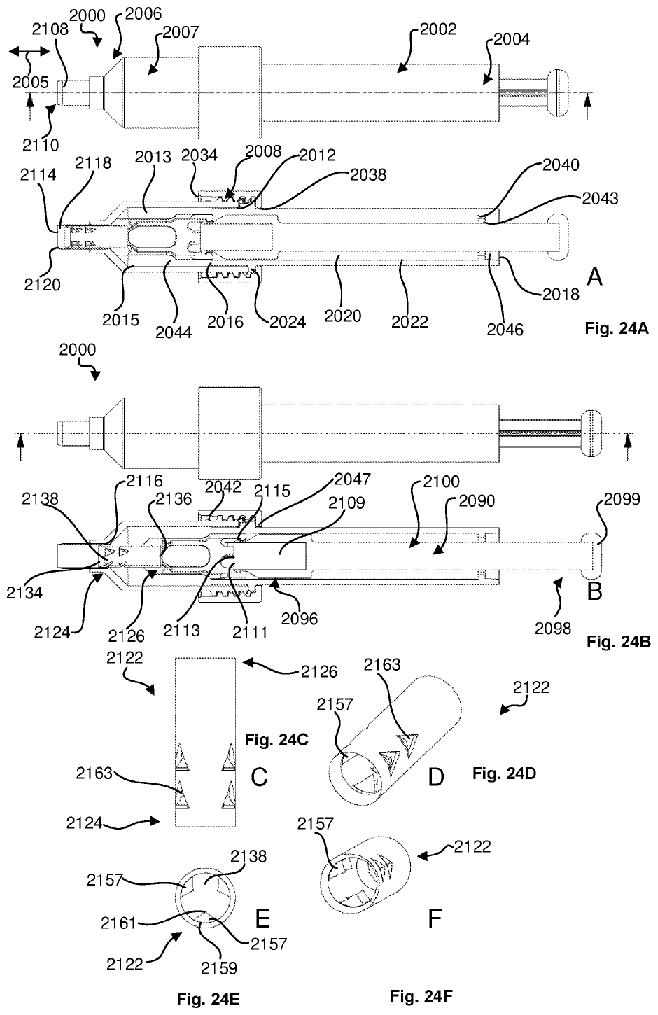

An example of a shark tooth variation for the first device is shown in FIGS. 24A to 24F. The device comprises a container 2002, a cutting element 2108 which may be a cylindrical blade, a tissue engagement component 2122 and plunger 2090 received in the container and movable longitudinally and along a length of the container. The plunger 2090 may be coupled to the cutting element and/or the tissue engagement component to allow them to be retracted into the container.

The tissue engagement component 2122 comprises a tapered projection 2157. In particular, the tissue engagement component 2122 comprises a plurality of tapered projections 2157. One or more of the tapered projections 2157 are configured to engage the tissue cut by The tissue engagement component 2122 comprises one or more of the tapered projections 2157 which taper from a base 2159 to a point 2161. One or more of the tapered projections 2157 project into the tissue engagement component lumen 2138. That is, one or more of the tapered projections 2157 project radially inwards. The one or more of the tapered projections 2157 project radially inwards towards a central axis of the tissue engagement component 2112.

The tissue engagement component 2112 comprises a tissue engagement opening 2163. In particular, the tissue engagement component 2122 comprises a plurality of tissue engagement openings 2163. One or more of the tissue engagement openings 2163 are formed in a wall of the tissue engagement component 2112. One or more of the tissue engagement openings 2163 extend through the wall of the tissue engagement component 2112. One or more of the tissue engagement openings 2163 are fluidly connected to the tissue engagement component lumen 2138. In some embodiments, the profile of one or more of the tissue engagement openings 2163 corresponds with that of a tapered projection 2157. That is, an outer profile of the relevant tapered projection 2157 is the same as a peripheral profile of the relevant tissue engagement openings 2163. The tissue engagement component 2122 and the cutting element 2108 may be separate components. That is, the tissue engagement component 2122 and the cutting element 2108 may be independently connectable to the plunger 2090. The plunger may comprise a sealing component which seals with an internal chamber of the container to generate a negative pressure or vacuum and cause suction to start the flow of fluid, pus or abscess contents into the container when the plunger and tissue engagement component are retracted.

In some variations, the tissue engagement component may be integrated with the cutting element as one piece, for example the cutting element may be cylindrical and may define a cutting lumen between a skin cutting distal edge of the cutting element and a second or proximal edge of the cutting element; the cutting element may have a plurality of shark teeth as described above (apertures and tapered projections pointing radially inward and to the proximal end). The tapered projections hook and pull on tissue when the cutting element is retracted so as to pull the skin plug back into the container. In other respects, the first device according to these variations may similar or the same as the device described above in Parts 1 and 2 of the description.

All of the features of the various embodiments disclosed in this specification (including any accompanying claims, abstract and drawings) may be combined in any combination, except combinations where at least some of such features are mutually exclusive.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1.-87. (canceled)

88. A device for removing tissue above a subcutaneous skin abscess, the device comprising:
a harpoon shaped tissue engagement component, comprising:
a shaft;
a penetration portion at a distal end of the shaft; and
a barb positioned on the shaft behind the penetration portion;
a container configured to contain at least part of the tissue engagement component; and
a cutting element comprising a cylindrical cutting sharp configured to cut through the skin surface of a patient to cut out a skin plug from epidermal and dermal tissue of a patient,
wherein the barb of the tissue engagement component is configured to engage the skin plug cut by the cutting element and penetrated by the penetration portion, such that movement of the tissue engagement component into a containment volume of the container causes movement of at least part of the skin plug cut by the cutting element into the containment volume, and
wherein the cylindrical cutting sharp defines a cutting lumen, the tissue engagement component extends within the cutting lumen of the cylindrical cutting sharp and there is a clearance between the shaft of the tissue engagement component and an inner diameter of the cylindrical cutting sharp.

89. The device of claim 88 wherein the clearance between the shaft of the tissue engagement component and an inner diameter of the cylindrical cutting sharp is 1 mm-3 mm.

90. The device of claim 88, wherein the tissue engagement component projects through the cutting lumen such that part of the tissue engagement portion is exposed beyond the cutting lumen, whereby the penetration portion is configured to penetrate the skin and tissue of a patient, prior to cutting of the skin plug by the cutting element.

91. The device of claim 88, further comprising a plunger configured move in a longitudinal direction of the container, wherein:
the container is configured to contain at least part of the plunger; and
movement of the plunger causes movement of the tissue engagement component.

92. The tissue retrieval device of claim 91 wherein the plunger is associated with a sealing member and movement of the plunger to retract the tissue engagement component into the container causes retraction of the sealing member to generate a negative pressure or vacuum in the container so as to draw subcutaneous fluid or contents of a subcutaneous skin abscess into the container.

93. The tissue retrieval device of claim 91, wherein movement of the plunger causes movement of the cutting element and wherein the tissue engagement component and the cutting element are connected to the plunger.

94. The device of claim 91, wherein the plunger is configured to be moved between:
a first position where at least part of the tissue engagement component and at least part of the cutting element are exposed; and
a second position where the tissue engagement component and the cutting element are contained within the containment volume of the container.

95. The device of claim 91, further comprising a locking element that is configured to engage with the plunger to inhibit movement of the plunger.

96. The device of claim 88, wherein:
the tissue engagement component defines a tissue engagement component lumen that extends between a first tissue engagement component opening and a second tissue engagement component opening; and
the tissue engagement component defines a barb opening that is fluidly connected to the tissue engagement component lumen.

97. The device of claim 88, wherein:
the container defines the containment volume and a distal volume;
the container comprises a container rim that extends inwardly in a radial direction of the container to define an inner container hole;
the container rim is between the containment volume and the distal volume; and
the inner container hole fluidly connects the containment volume and the distal volume.

98. The device of claim 97, further comprising a first plunger configured move in a longitudinal direction of the container, wherein:
the tissue retrieval device further comprises a second plunger configured to connect to the first plunger such that, when connected, movement of the second plunger causes corresponding movement of the first plunger;
wherein the second plunger is configured to be moved between:
an initial position where the second plunger is connected to a first plunger connection portion of the first plunger; and a plunger connection position where the second plunger is connected to a second plunger connection portion of the first plunger; and
the plunger connection position and a retracted position.

99. The device of claim 88, further comprising a plunger sleeve comprising:
a first sleeve connector member; and
a second sleeve connector member,
wherein the plunger sleeve is configured to contain at least part of the first plunger and at least part of the second plunger within a plunger sleeve volume,
wherein the first sleeve connector member is configured to be moved between:
a first position where the first sleeve connector member is configured to abut an end of the first plunger to inhibit movement of the first plunger with respect to the plunger sleeve; and
a second position where the first sleeve connector member is configured to enable movement of the first plunger with respect to the plunger sleeve.

100. The device of claim 88, wherein the cutting element has a length of 4 mm to 10 mm and an outer diameter of 3 mm-6 mm.

101. The device of claim 88, wherein the tissue engagement component shaft has a diameter of 1 mm to 3 mm.

102. The device of claim 88, wherein the tissue engagement component is positioned centrally within the lumen of the cylindrical cutting sharp.

103. A percutaneous drainage device for draining a subcutaneous skin abscess, the device comprising:
a cannula defining a cannula lumen that extends between a first cannula opening and a second cannula opening; and
a penetration component configured to be slidably received within the cannula, the penetration component comprising:
a penetration portion configured to penetrate tissue of a patient, thereby enabling introduction of the first cannula opening to a subcutaneous fluid collection site; and
a penetration component lumen that extends between a first penetration component opening and a second penetration component opening, the penetration component lumen being configured to receive a guide wire,
wherein the device further comprises a penetration component cap configured to removably engage with the penetration component to enable selective obstruction of the second penetration component opening.

104. The percutaneous drainage device of claim 103, wherein:
the cannula comprises a recess; and
the penetration component comprises a projecting feature, and
wherein the recess is configured to receive the projecting feature when the penetration component is received within the cannula, thereby impeding rotation of the cannula with respect to the penetration component.

105. The percutaneous drainage device of claim 103, further comprising a cap defining a cap lumen that extends from a first cap opening to a second cap opening, the cap lumen being configured to receive the cannula and the penetration component,
wherein the cap comprises a cap groove that extends from the first cap opening to the second cap opening, the cap groove being configured to enable the passage of guide wire from the cap lumen to outside of the cap.

106. The percutaneous drainage device of claim 103, further comprising a collar configured to:
removably engage with the cannula; and
be movable in a longitudinal direction of the cannula.

107. The percutaneous drainage device of claim 103, further comprising a connecting component comprising:
a collar engaging portion configured to removably engage with the collar; and
an extension portion extending radially from the collar contacting portion, the extension portion being configured to connect to the tissue of the patient, wherein:
the cannula defines a cannula thread; and
the collar comprises a collar lumen configured to receive the cannula and to cooperate with the cannula thread such that:
rotation of the collar with respect to the cannula causes movement of the collar in the longitudinal direction of the cannula; and
movement of the collar in the longitudinal direction of the cannula is impeded without rotation of the collar with respect to the cannula.
